(19) United States
(12) Patent Application Publication
(10) Pub. No.: US 2011/0305639 A1
(43) Pub. Date: Dec. 15, 2011

(54) METHOD AND FORMULATION FOR REDUCING AGGREGATION OF A MACROMOLECULE UNDER PHYSIOLOGICAL CONDITIONS

(76) Inventors: Brian Lobo, Carlsbad, CA (US); Sabrina Lo, San Francisco, CA (US); Yuchang John Wang, Los Altos, CA (US); Rita L. Wong, Redwood City, CA (US)

(21) Appl. No.: 13/107,137

(22) Filed: May 13, 2011

Related U.S. Application Data
(63) Continuation of application No. PCT/US09/64610, filed on Nov. 16, 2009.
(60) Provisional application No. 61/115,441, filed on Nov. 17, 2008.

(51) Int. Cl. A61K 31/724; A61K 39/395; A61K 49/00; A61P 35/00; A61P 35/02; A61P 37/00; A61P 19/02; A61P 25/00; A61P 1/00; A61P 1/04; A61P 21/04; A61P 17/06; G01N 33/487; G01J 3/00; A61K 38/16

(52) U.S. Cl. 424/9.1; 514/58; 514/1.1; 424/130.1; 424/173.1; 424/133.1; 73/53.01; 356/51

(57) ABSTRACT

The invention provides a method for reducing aggregation and inhibiting flocculation of a macromolecule, such as a protein, under physiological conditions, by the addition of certain cyclodextrins (CDs). The invention also provides a method to minimize inflammation at the injection site during subcutaneous administration of a macromolecule and pharmaceutical formulations for such administration. Further the invention provides methods of treating a CD20 positive cancer or an autoimmune disease, comprising administering a humanized anti-CD20 antibody in a pharmaceutical formulation of the invention. The invention further provides an in vitro dialysis method to evaluate the ability of an excipient to reduce aggregation of an antibody or other macromolecule under physiological conditions.

### PRIORITY TO RELATED APPLICATIONS

This is a continuation application of International Application No. PCT/US2009/064610, filed Nov. 16, 2009 claiming benefit from provisional application 61/115,441 filed Nov. 17, 2008, the contents of which are incorporated herein by reference.

### REFERENCE TO SEQUENCE LISTING SUBMITTED AS A TEXT FILE VIA EFS-WEB

A sequence listing is submitted concurrently with the specification as an ASCII formatted text file via EFS-Web, with a file name of “P2391R1C1Sequence.txt”, a creation date of May 12, 2011, and a size of 40 kilobytes. The sequence listing filed via EFS-Web is part of the specification and is hereby incorporated by reference in its entirety herein.

### FIELD OF THE INVENTION

The invention relates to a method to minimize inflammation at the injection site for subcutaneous administration of a macromolecule by reducing aggregation under physiological conditions.

### BACKGROUND OF THE INVENTION

In the past two decades, recombinant DNA technology has led to a significant increase in the number of medicines which are biomolecules, in particular, proteins. The increase in biomolecule medications has led to new challenges in drug formulation. High doses of protein therapeutics such as antibodies can be delivered to the patient by intravenous infusion but this route of drug administration is inconvenient and it is generally preferable to formulate the protein therapeutic for subcutaneous injection where possible. However, the drug solution for subcutaneous injection is at a much smaller volume than for i.v. infusion so the protein is necessarily present at a higher concentration. At high therapeutic protein concentrations of tens of milligrams per milliliter it is important to keep the therapeutic proteins stably dissolved for extended periods of time. High concentration solutions of proteins increase the likelihood of protein-protein interactions favoring aggregation; prevention of aggregation has become a major issue for protein drug formulation. Aggregation leads to a number of problems, including decreased bioavailability of the active protein, altered pharmacokinetics, and unwanted immunogenicity. (Frokjaer, S, and Otzen, D. E., Nat. Rev. Drug. Discov. 4: 298-306 (2005); Jiskoot, W. and Crommelin, D. J. A., EJHP Practice 12:20-21 (2006)).

The prevention of aggregation remains largely empirical, as the molecular details of the aggregation process are generally unknown. A typical strategy is to add stabilizers to a protein solution. Commonly used stabilizers include sugars, salts, free amino acids such as L-arginine and L-glutamine (Golovanov, A. P. et al., J. Am. Chem. Soc. 126:8933-8939 (2004)), polyols (Singh, S, and Singh, J., AAPS Pharm. Sci. Tech 4: 1-9 (2003); Mishra, R. et al., J. Biol. Chem. 280:15553-15560 (2005)), polyethylene glycols (PEGs), and other polymers, such as polysorbates or poloxamers that may reduce protein-protein interactions (Frokjaer and Otzen, supra; Lee, R. C. et al., Ann. Biomed. Eng. 34: 1190-1200 (2006); (Nema, S. et al., PDA Journal of Pharmaceutical Science and Technology 51: 166-171 (1997)).

Cyclodextrins (CDs) are cyclic oligosahccharides having d-glucopyranose units linked with alpha-(1,4) glycosidic bonds. CDs are produced from corn or other starches through the action of the amylase, cyclodextrin transglucosylase. The most common naturally occurring cyclodextrins are alpha-cyclodextrin, beta-cyclodextrin and gamma-cyclodextrin, consisting of 6, 7 and 8 glucopyranose units, respectively. As natural cylcodextrins, beta-cyclodextrin in particular, have low aqueous solubility, a number of derivatives having improved solubility and other physicochemical properties have been synthesized. Commercially available CD derivatives include methylated CDs, 2-hydroxypropylated CDs, acetylated CDs, branched CDs and sulfobutyl CDs. Synonyms for cyclodextrin include Cavitron, cyclic oligosaccharide, cycloamulose, and cycloglucan. (For review, see Loftsson, T., and Brewster, M. E., J. Pharm. Sci. 85:101-(1996); Uekama, K. et al., Chem. Rev. 98:2045-2076 (1998); Irie, T. and Uekama, K., Advanced Drug Delivery Reviews 36:101-123 (1999); and Szjetli, J., Pure Appl. Chem. 76:1825-1845 (2004)). Cyclodextrins are less than 25,000 daltons in molecular weight and hence can be removed from systemic circulation by glomerular filtration and are not expected to accumulate in the body. The lack of toxicity of natural cyclodextrins as well as a number of pharmaceutically relevant derivatives such as hydroxypropyl-beta-CD and sulfobutyl-beta-CD has been well documented (Uekama et al., supra; Szjetli, supra).

Cyclodextrins take the shape of a truncated cone where the interior is hydrophobic and exterior is hydrophilic. The hydrophobic cavity provides an environment into which appropriately sized non-polar compounds can be included and form complexes. CD and its derivatives have been used as solubilizers for poorly water soluble drugs. For example, itraconazole (Sporanox™) is solubilized with hydroxypropyl beta-CD and ziprasidone mesylate (Geodon™) is solubilized with sulfobutyl ether beta-CD. Other uses of CD include drug stabilization, taste masking, and adsorbent for essential oils. Currently available drug products that contain cyclodextrin include Sporanox™ (Janssen, Belgium), Prostavasin™ (Ono, Japan; Schwarz, Germany), Prostandin 500™ (Ono, Japan), Geodon™ (Pfizer, USA), VFEND™ (Pfizer, USA), MitoExtra Mitozytrex™ (Novartis, Switzerland), and Voltaren™ (Novartis, Switzerland). See also Table 1 of Szjetli, supra. These formulations are all limited to small molecule compounds.

Large drug molecules such as peptides and proteins can also form complexes with cyclodextrins. The improved bioavailability of peptide drugs complexed with CDs is thought to stem in part from the inhibitory effect of CDs on cellular efflux pumps (Challa, R. et al., AAPS Pharm. Sci. Tech. 6:E329-357 (2005)). The mechanism of stabilization for proteins and peptides is also qualitatively different than in the case of small molecule drugs. While CDs can form inclusion complexes with small molecule drugs, CDs appear to bind to specific solvent-exposed amino acid residues of the protein or peptide (Aachmann, F. L. et al., Protein Engineering 16:905-912 (2003)). Maximum benefit is usually obtained at low cyclodextrin concentrations, and the benefits are often only partially concentration dependent. For example, aggregation of IL-2 was optimally inhibited by 0.5% HP-beta-cyclodextrin. (Loftsson and Brewster, supra). The solubility of human growth hormone was improved by CDs present at about 2-6%, with alpha and gamma CDs found to be several-fold less effective than beta CDs. (Otzen, D. E. et al., Protein Sci. 11:1779-1787 (2002)).

The CD20 antigen (also called human B-lymphocyte-restricted differentiation antigen, Bp35) is a hydrophobic transmembrane protein with a molecular weight of approximately 35 kD located on pre-B and mature B lymphocytes (Valentine et al., J. Biol. Chem. 264(19):11282-11287 (1989); and Einfeld et al., EMBO J. 7(3):711-717 (1988)). The antigen is also expressed on greater than 90% of B cell non-Hodgkin's lymphomas (NHL) (Anderson et al., Blood 63(6):1424-1433 (1984)), but is not found on hematopoietic stem cells, pro-B cells, normal plasma cells or other normal tissues (Tedder et al., J. Immunol. 135(2):973-979 (1985)). CD20 is thought to regulate an early step(s) in the activation process for cell cycle initiation and differentiation (Tedder et al., supra) and possibly functions as a calcium ion channel (Tedder et al., J. Cell. Biochem. 14D:195 (1990)).

Given the expression of CD20 in B cell lymphomas, this antigen has been a useful therapeutic target to treat such lymphomas. For example, the rituximab (RITUXAN®, MABTHERA®) antibody, which is a genetically engineered chimeric murine/human monoclonal antibody directed against human CD20 antigen (commercially available from Genentech, Inc., South San Francisco, Calif., U.S. and F. Hoffmann-La Roche A G, Basel, Switzerland), is used for the treatment of patients with relapsed or refractory low-grade or follicular, CD20 positive, B cell non-Hodgkin's lymphoma. Rituximab is the antibody referred to as “C2B8” in U.S. Pat. No. 5,736,137 issued Apr. 7, 1998 (Anderson et al.) and in U.S. Pat. No. 5,776,456. Other anti-CD20 antibodies indicated for the treatment of NHL include the murine antibody Zevalin™ which is linked to the radioisotope Yttrium-90 (IDEC Pharmaceuticals, San Diego, Calif.), and Bexxar™ which is a another fully murine antibody conjugated to 1-131 (Corixa, Wash.).

CD20 is also a useful target antigen for treating autoimmune diseases. Rituximab has also been studied in a variety of non-malignant autoimmune disorders, in which B cells and autoantibodies appear to play a role in disease pathophysiology, including Edwards et al., Biochem Soc. Trans. 30:824-828 (2002). Rituximab has been reported to potentially relieve signs and symptoms of, for example, rheumatoid arthritis (RA) (Leandro et al., Ann. Rheum. Dis. 61:883-888 (2002); Edwards et al., Arthritis Rheum., 46 (Suppl. 9): S46 (2002); Stahl et al., Ann. Rheum. Dis., 62 (Suppl. 1): OP004 (2003); Emery et al., Arthritis Rheum. 48(9): 5439 (2003)), lupus (Eisenberg, Arthritis. Res. Ther. 5:157-159 (2003); Leandro et al. Arthritis Rheum. 46: 2673-2677 (2002); Gorman et al., Lupus, 13: 312-316 (2004)), immune thrombocytopenic purpura (D'Arena et al., Leuk. Lymphoma 44:561-562 (2003); Stasi et al., Blood, 98: 952-957 (2001); Saleh et al., Semin. Oncol., 27 (Supp 12):99-103 (2000); Zaia et al., Haematolgica, 87: 189-195 (2002); Ratanatharathorn et al., Ann. Int. Med., 133: 275-279 (2000)), pure red cell aplasia (Auner et al., Br. J. Haematol., 116: 725-728 (2002)); autoimmune anemia (Zaja et al., Haematologica 87:189-195 (2002) (erratum appears in Haematologica 87:336 (2002)), cold agglutinin disease (Layios et al., Leukemia, 15: 187-8 (2001); Berentsen et al., Blood, 103: 2925-2928 (2004); Berentsen et al., Br. J. Haematol., 115: 79-83 (2001); Bauduer, Br. J. Haematol., 112: 1083-1090 (2001); Damiani et al., Br. J. Haematol., 114: 229-234 (2001)), type B syndrome of severe insulin resistance (Coll et al., N. Engl. J. Med., 350: -311 (2004), mixed cryoglobulinemia (DeVita et al., Arthritis Rheum. 46 Suppl. 9:S206/S469 (2002)), myasthenia gravis (Zaja et al., Neurology, 55: 1062-63 (2000); Wylam et al., J. Pediatr., 143: 674-677 (2003)), Wegener's granulomatosis (Specks et al., Arthritis & Rheumatism 44: 2836-2840 (2001)), refractory pemphigus vulgaris (Dupuy et al., Arch Dermatol., 140:91-96 (2004)), dermatomyositis (Levine, Arthritis Rheum., 46 (Suppl. 9):S1299 (2002)), Sjogren's syndrome (Somer et al., Arthritis & Rheumatism, 49: 394-398 (2003)), active type-II mixed cryoglobulinemia (Zaja et al., Blood, 101: 3827-3834 (2003)), pemphigus vulgaris (Dupay et al., Arch. Dermatol., 140: 91-95 (2004)), autoimmune neuropathy (Pestronk et al., J. Neurol. Neurosurg. Psychiatry 74:485-489 (2003)), paraneoplastic opsoclonus-myoclonus syndrome (Pranzatelli et al. Neurology 60(Suppl. 1) PO5.128:A395 (2003)), and relapsing-remitting multiple sclerosis (RRMS). Cross et al. (abstract) “Preliminary results from a phase II trial of Rituximab in MS” Eighth Annual Meeting of the Americas Committees for Research and Treatment in Multiple Sclerosis, 20-21 (2003).

The present invention provides methods and formulations for preventing the aggregation of macromolecules, such as antibodies, under physiological conditions. The methods of the invention offer advantages in the preparation of formulations of therapeutic proteins such as the anti-CD20 antibodies described in the specification. These advantages include the ability to prepare formulations for subcutaneous injection that will provide increased bioavailablity of the therapeutic antibody and decreased inflammation at the injection site, as well as additional advantages that will be apparent from the detailed description below.

### SUMMARY OF THE INVENTION

Cyclodextrins have been used by biochemists as solubilizers for poorly water soluble drugs. Our findings that different types of cyclodextrins (e.g., sulfo-butyl ether, hydroxy propyl gamma, hydroxy propyl beta) inhibited aggregation and flocculation of a protein, in particular, an antibody, is unexpected as antibodies are highly water soluble. The finding that cyclodextrins inhibited aggregation and flocculation of an antibody at high concentrations therefore represents a novel use for cyclodextrin. We have also developed a novel in vitro screening method which includes the use of dialysis tubing with defined molecular weight (MW) cut-off and customized release media, both of which mimic the physiological conditions at the injection site.

The invention provides a method for reducing aggregation and inhibiting flocculation of a macromolecule, such as a protein, under physiological conditions, by the addition of 2% to 30% cyclodextrins (CDs), where the cyclodextrin is selected from the group consisting of hydroxy propyl beta (HP-Beta), hydroxy propyl gamma (HP-Gamma) and sulfo-butyl ether (SBE) cyclodextrin. The significant reduction in aggregation and flocculation by the addition of CDs also correlated to a significant reduction in inflammation at the site of subcutaneous injection in rats. The invention further provides a method to minimize inflammation at the injection site during subcutaneous administration of a macromolecule, such as a protein, by the addition of 2% to 30% HP-Beta cyclodextrin, HP-Gamma cyclodextrin, or SBE cyclodextrin to the subcutaneous formulation. In various embodiments of the invention, the macromolecule is an antibody. In further embodiments of the invention the antibody is a therapeutic antibody or a diagnostic antibody.

In various embodiments of the invention, the macromolecule is an anti-CD20 antibody. In certain embodiments of the invention, the anti-CD20 antibody is a humanized antibody. In certain embodiments of the invention, the anti-CD20 antibody comprises one of the variants A, B, C, D, F, G, H or I from Table 1. The invention further provides methods and formulations wherein the anti-CD20 antibody comprises an amino acid sequence selected from the group consisting of SEQ ID NO:1-15. In further embodiments of the invention, the antibody comprises the light chain variable domain of SEQ ID NO:1 and the heavy chain variable domain of SEQ ID NO:2, or the light chain variable domain of SEQ ID NO:3 and the heavy chain variable domain of SEQ ID NO:4, or the light chain variable domain of SEQ ID NO:3 and the heavy chain variable domain of SEQ ID NO:5. The invention further provides methods and formulations wherein the antibody comprises the full-length light chain of SEQ ID NO:6 and the full-length heavy chain of SEQ ID NO:7, SEQ ID NO:8, or SEQ ID NO:15. The invention further provides methods and formulations wherein the antibody comprises the full-length light chain of SEQ ID NO:9 and the full-length heavy chain of SEQ ID NO:10, SEQ ID NO:11, SEQ ID NO:12, SEQ ID NO:13, or SEQ ID NO:14.

In further aspects, the invention provides a pharmaceutical formulation for subcutaneous administration of a macromolecule, such as a protein, comprising 2% to 30% HP-Beta cyclodextrin, HP-Gamma cyclodextrin, or SBE cyclodextrin. In some embodiments, the invention provides a pharmaceutical formulation for subcutaneous administration of an antibody comprising an antibody at a concentration range of 10 mg/ml to 200 mg/ml, and 2% to 30% HP-Beta cyclodextrin, HP-Gamma cyclodextrin, or SBE cyclodextrin. In certain embodiments, the antibody concentration range is from 30-150 mg/ml. In further embodiments, the antibody concentration range is from 100-150 mg/ml. In certain embodiments, the pharmaceutical formulation comprises HP-Beta cyclodextrin at a concentration of 5% to 30%. In certain embodiments, the pharmaceutical formulation comprises HP-Gamma cyclodextrin at a concentration of 5% to 20%. In certain embodiments, the pharmaceutical formulation further comprises arginine succinate at a concentration of 50 mM to 200 mM. In certain embodiments, the pharmaceutical formulation comprises SBE cyclodextrin at a concentration of 2% to 9%. In certain embodiments, the pharmaceutical formulation comprises an antibody at a concentration of about 100 mg/ml and HP-Beta cyclodextrin at a concentration of 15% to 30%. In certain embodiments, the pharmaceutical formulation comprises an antibody at a concentration of about 150 mg/ml and HP-Beta cyclodextrin at a concentration of about 30%. In certain embodiments, the pharmaceutical formulation comprises an antibody at a concentration of about 150 mg/ml and HP-Gamma cyclodextrin at a concentration of about 10%. In certain embodiments, the pharmaceutical formulation further comprises arginine succinate at a concentration of 50 mM to 200 mM. In a specific embodiment, the pharmaceutical formulation comprises a humanized 2H7 antibody at a concentration range of 100 mg/ml to 150 mg/ml, HP-Gamma cyclodextrin at a concentration of 15% to 30%, and arginine succinate at a concentration of 50 mM to 100 mM. In further embodiments, the pharmaceutical composition further comprises 30 mM sodium acetate; 5% trehalose dihydrate; and 0.03% Polysorbate 20, at pH 5.3.

The invention further provides any of the above formulations comprising a humanized anti-CD20 antibody consisting of any of the antibodies listed in Table 1. The invention further provides formulations wherein the anti-CD20 antibody comprises an amino acid sequence selected from the group consisting of SEQ ID NO:1-15. In further embodiments of the invention, the antibody comprises the light chain variable domain of SEQ ID NO:1 and the heavy chain variable domain of SEQ ID NO:2, or the light chain variable domain of SEQ ID NO:3 and the heavy chain variable domain of SEQ ID NO:4. The invention further provides methods and formulations wherein the antibody comprises the full-length light chain of SEQ ID NO:6 and the full-length heavy chain of SEQ ID NO:7, SEQ ID NO:8, or SEQ ID NO:15. The invention further provides methods and formulations wherein the antibody comprises the full-length light chain of SEQ ID NO:9 and the full-length heavy chain of SEQ ID NO:10, SEQ ID NO:11, SEQ ID NO:12, SEQ ID NO:13, or SEQ ID NO:14.

The invention further provides a method of treating a cancer of CD20 expressing B cells comprising administering any one of the humanized anti-CD20 antibodies of Table 1 in a pharmaceutical formulation comprising 2% to 30% HP-Beta cyclodextrin, HP-Gamma cyclodextrin, or SBE cyclodextrin. The CD20 positive B cell cancer is preferably a B cell lymphoma or leukemia. In specific embodiments, formulations comprising the humanized 2H7 antibodies that bind human CD20 (hCD20) and functional fragments thereof are used to treat non-Hodgkin's lymphoma (NHL), indolent NHL including relapsed indolent NHL and rituximab-refractory indolent NHL, lymphocyte predominant Hodgkin's disease (LPHD), small lymphocytic lymphoma (SLL), chronic lymphocytic leukemia (CLL). In specific embodiments, formulations comprising humanized CD20 binding antibodies, in particular, variants A, B, C, D or H from Table 1, or functional fragments thereof, are used to treat the CD20 positive B cell cancers listed above.

The invention also provides a method of treating an autoimmune disease, comprising administering to a patient suffering from the autoimmune disease, a therapeutically effective amount of a humanized 2H7 antibody of Table 1 in a pharmaceutical formulation comprising 2% to 30% HP-Beta cyclodextrin, HP-Gamma cyclodextrin, or SBE cyclodextrin. In specific embodiments, the autoimmune disease is selected from the group consisting of rheumatoid arthritis (RA) and juvenile rheumatoid arthritis, and the RA patients are methotrexate (Mtx)-inadequate responders and TNFα-antagonist inadequate responders, rituximab-refractory or relapse patients. In one embodiment, an RA patient is refractory or relapsed with respect to another anti-CD20 therapeutic antibody. In other embodiments, the autoimmune disease is selected from the group consisting of systemic lupus erythematosus (SLE) including lupus nephritis, multiple sclerosis (MS), including relapsing remitting multiple sclerosis (RRMS), Wegener's disease, inflammatory bowel disease, ulcerative colitis, idiopathic thrombocytopenic purpura (ITP), thrombotic thrombocytopenic purpura (TTP), autoimmune thrombocytopenia, multiple sclerosis, psoriasis, IgA nephropathy, IgM polyneuropathies, myasthenia gravis, ANCA associated vasculitis, diabetes mellitus, Reynaud's syndrome, Sjogren's syndrome, Neuromyelitis Optica (NMO) and glomerulonephritis. In specific embodiments, formulations comprising humanized CD20 binding antibodies, in particular, variants A, B, C, D or H from Table 1, or functional fragments thereof, are used to treat the autoimmune diseases listed above.

In certain embodiments of the methods of treating the aforementioned diseases, the subject or patient suffering from the disease is a primate, preferably a human.

The invention further provides a method of improving or maintaining solubilization of or minimizing precipitation of an antibody in an aqueous subcutaneous formulation upon injection at the injection site of a patient, comprising adding 2% to 30% HP-Beta cyclodextrin, HP-Gamma cyclodextrin, or SBE cyclodextrin to the aqueous subcutaneous formulation. In certain embodiments, the pharmaceutical formulation comprises HP-Beta cyclodextrin at a concentration of 5% to 30%. In certain embodiments, the pharmaceutical formulation comprises HP-Gamma cyclodextrin at a concentration of 5% to 20%. In certain embodiments, the pharmaceutical formulation further comprises arginine succinate at a concentration of 50 mM to 200 mM. In certain embodiments, the pharmaceutical formulation comprises SBE cyclodextrin at a concentration of 2% to 9.

The invention further provides a method of increasing the bioavailability of an antibody to be administered subcutaneously, comprising adding 2% to 30% HP-Beta cyclodextrin, HP-Gamma cyclodextrin, or SBE cyclodextrin to an aqueous subcutaneous formulation comprising the antibody. In certain embodiments, the pharmaceutical formulation comprises HP-Beta cyclodextrin at a concentration of 5% to 30%. In certain embodiments, the pharmaceutical formulation comprises HP-Gamma cyclodextrin at a concentration of 5% to 20%. In certain embodiments, the pharmaceutical formulation further comprises arginine succinate at a concentration of 50 mM to 200 mM. In certain embodiments, the pharmaceutical formulation comprises SBE cyclodextrin at a concentration of 2% to 9.

The invention further provides an in vitro dialysis method to evaluate the ability of an excipient to reduce aggregation of an antibody or other macromolecule under physiological conditions, comprising: dialyzing formulations of the macromolecule with and without the test excipient against a test medium to simulate physiologic conditions at 37° C. with constant agitation; sampling the modified media solution; and measuring the appearance such as turbidity of the samples and the amount of protein present in the release medium were measured by methods such as a UV photometric scan, wherein increased protein concentration and decreased turbidity in the release medium in the assay containing the test excipient as compared to the control lacking excipient are indicative of the ability of the test excipient to reduce aggregation of the macromolecule. In specific embodiments the media relates to a modified PBS solution such as containing 167 mM Sodium, 140 mM Chloride, 17 mM Phosphate, 4 mM Potassium. In specific embodiments of the method, the dialysis tubing has a 1 million Dalton molecular weight cut-off. In further specific embodiments of the method, protein concentration and turbidity in the test samples are measured using UV spectrometry. In further embodiments of the method, the method includes visually inspecting the modified release medium and the solution inside the dialysis tubing for precipitation, wherein decreased precipitation in the dialysis tubing containing the test excipient as compared to the control lacking excipient is indicative of the ability of the test excipient to reduce aggregation of the macromolecule.

### BRIEF DESCRIPTION OF THE FIGURES

FIG. 1 shows the aggregation of 2H7 under physiological conditions. 2H7 at 150 mg/ml was dialysed into PBS for two days at 37° C.

FIG. 2 shows the in vitro dialysis model used to evaluate the effects of excipients on 2H7 aggregation under physiological conditions. A 250 ml glass jar is filled with 220 ml of modified PBS solution (167 mM Sodium, 140 mM Chloride, 17 mM Phosphate, 4 mM Potassium) at 37° C. A 6 cm length of 12 mm dialysis tubing is clamped at one end, filled with approximately 1 ml of test sample, excess air is removed, and the other end of the tubing is clamped to the seal. The jar is placed at 37° C. with constant stirring.

FIG. 3 shows the behavior of the controls in the in vitro dialysis model. Both 2H7 and rhuMab CD11a were tested in the model shown in FIG. 2. The cumulative percentage of protein released into the PBS solution was measured at 2.5, 6, 12, 24, 33 and 48 hour timepoints.

FIG. 4 shows the effect of 2-9% SBE-cyclodextrin on the release of 2H7 in the in vitro model.

FIG. 5 shows the effect of 5-20% HP-Gamma-cyclodextrin on the release of 2H7 in the in vitro model.

FIG. 6 shows the effect of 5-20% HP-Beta-cyclodextrin on the release of 2H7 in the in vitro model.

FIG. 7 shows the effect of HP-Gamma-cyclodextrin and arginine succinate on the release of 2H7 in the in vitro model.

### DETAILED DESCRIPTION OF THE EMBODIMENTS

The various forms of the verb “to aggregate” refer to a process whereby individual protein molecules or complexes associate to form aggregates. An “aggregate” is a polymeric assembly comprising molecules or complexes of protein. Aggregation can proceed to the extent that a visible precipitate is formed. The formation of such a visible precipitate is also referred to herein as “flocculation.”

The relative amount of precipitation of a macromolecule may be determined, for example, by comparison to a visual control. Additional methods of assaying precipitation are known in the art and described below, e.g., the in vitro dialysis method described in detail in Example 2, or the in vivo model described in Example 3.

The term “bioavailability” refers to the degree to which or rate at which a drug or other substance is absorbed or becomes available at the site of physiological activity after administration. The bioavailability of a macromolecule may be assayed by in vivo pharmacokinetics methods known in the art.

The term “macromolecule” refers to a molecule with a molecular weight of at least 10,000 daltons, and may include proteins, such as antibodies.

The terms “excipient” or “pharmaceutical excipient” refer to compounds which may decrease aggregation of a macromolecule. Excipients may include sugars, salts, free amino acids such as L-arginine and L-glutamine, polyols, polyethylene glycols (PEGs), and other polymers, such as polysorbates, poloxamers, or polyvinylpyrrolidone.

The term “cyclodextrin” (or “CD”) refers to cyclic oligosaccharides having d-glucopyranose units linked with alpha-(1,4) glycosidic bonds. The most common naturally occurring cyclodextrins are alpha-cyclodextrin, beta-cyclodextrin and gamma-cyclodextrin, consisting of 6, 7 and 8 glucopyranose units, respectively. Synonyms for cyclodextrin include Cavitron, cyclic oligosaccharide, cycloamulose, and cycloglucan. The term “cyclodextrin” as used herein, may further include cyclodextrin derivatives, including, but not limited to, methylated CDs, 2-hydroxypropylated CDs, acetylated CDs, branched CDs and sulfobutyl CDs.

The term “therapeutic antibody” refers to an antibody that is used in the treatment of disease. A therapeutic antibody may have various mechanisms of action. A therapeutic antibody may bind and neutralize the normal function of a target. For example, a monoclonal antibody that blocks the activity of the protein needed for the survival of a cancer cell causes the cell's death. Another therapeutic monoclonal antibody may bind and activate the normal function of a target. For example, a monoclonal antibody can bind to a protein on a cell and trigger an apoptosis signal. Finally, if a monoclonal antibody binds to a target expressed only on diseased tissue, conjugation of a toxic payload (effective agent), such as a chemotherapeutic or radioactive agent, to the monoclonal antibody can create an agent for specific delivery of the toxic payload to the diseased tissue, reducing harm to healthy tissue.

The term “diagnostic antibody” refers to an antibody that is used as a diagnostic reagent for a disease. The diagnostic antibody may bind to a target that is specifically associated with, or shows increased expression in, a particular disease. The diagnostic antibody may be used, for example, to detect a target in a biological sample from a patient, or in diagnostic imaging of disease sites, such as tumors, in a patient.

The “CD20” antigen is a non-glycosylated, transmembrane phosphoprotein with a molecular weight of approximately 35 kD that is found on the surface of greater than 90% of B cells from peripheral blood or lymphoid organs. CD20 is expressed during early pre-B cell development and remains until plasma cell differentiation; it is not found on human stem cells, lymphoid progenitor cells or normal plasma cells. CD20 is present on both normal B cells as well as malignant B cells. Other names for CD20 in the literature include “B-lymphocyte-restricted differentiation antigen” and “Bp35”. The CD20 antigen is described in, for example, Clark and Ledbetter, Adv. Can. Res. 52:81-149 (1989) and Valentine et al. J. Biol. Chem. 264(19):11282-11287 (1989).

The term “antibody” is used in the broadest sense and specifically covers monoclonal antibodies (including full length monoclonal antibodies), multispecific antibodies (e.g., bispecific antibodies), and antibody fragments so long as they exhibit the desired biological activity or function.

The biological activity of the humanized CD20 binding antibodies of the invention will include at least binding of the antibody to human CD20, more preferably binding to human and other primate CD20 (including cynomolgus monkey, rhesus monkey, chimpanzees). The antibodies would bind CD20 with a K_{d} value of no higher than 1×10⁻⁸, preferably a K_{d} value no higher than about 1×10⁻⁹, and be able to kill or deplete B cells in vivo, preferably by at least 20% when compared to the appropriate negative control which is not treated with such an antibody. B cell depletion can be a result of one or more of ADCC, CDC, apoptosis, or other mechanism. In some embodiments of disease treatment herein, specific effector functions or mechanisms may be desired over others and certain variants of the humanized 2H7 are preferred to achieve those biological functions, such as ADCC.

“Antibody fragments” comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981)), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol.340(5):1073-1093 (2004); Fellouse, Proc. Nat. Acad. Sci. USA 101(34):12467-12472 (2004); and Lee et al. J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806; 5,569,825; 5,591,669 (all of GenPharm); 5,545,807; WO 1997/17852; U.S. Pat. Nos. 5,545,807; 5,545,806; 5,569,825; 5,625,126; 5,633,425; and 5,661,016; Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995).

“Functional fragments” of the CD20 binding antibodies of the invention are those fragments that retain binding to CD20 with substantially the same affinity as the intact full length molecule from which they are derived and show biological activity including depleting B cells as measured by in vitro or in vivo assays such as those described herein.

The term “variable” refers to the fact that certain segments of the variable domains differ extensively in sequence among antibodies. The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As referred to herein, the “consensus sequence” or consensus V domain sequence is an artificial sequence derived from a comparison of the amino acid sequences of known human immunoglobulin variable region sequences. Based on these comparisons, recombinant nucleic acid sequences encoding the V domain amino acids that are a consensus of the sequences derived from the human K and the human H chain subgroup III V domains were prepared. The consensus V sequence does not have any known antibody binding specificity or affinity.

“Chimeric” antibodies (immunoglobulins) have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Humanized antibody as used herein is a subset of chimeric antibodies.

“Humanized” forms of non-human (e.g., murine) antibodies are chimeric antibodies which contain minimal sequence derived from non-human immunoglobulin. For the most part, humanized antibodies are human immunoglobulins (recipient or acceptor antibody) in which hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence although the FR regions may include one or more amino acid substitutions that improve binding affinity. The number of these amino acid substitutions in the FR are typically no more than 6 in the H chain, and in the L chain, no more than 3. The humanized antibody optionally also will comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al., J. Immunol. Methods 202:163 (1996), may be performed.

Throughout the present specification and claims, unless otherwise indicated, the numbering of the residues in the constant domains of an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody. The residues in the V region are numbered according to Kabat numbering unless sequential or other numbering system is specifically indicated.

CD20 antibodies include: “C2B8,” which is now called “rituximab” (“RITUXAN®”) (U.S. Pat. No. 5,736,137); the yttrium-[90]-labelled 2B8 murine antibody designated “Y2B8” or “Ibritumomab Tiuxetan” (ZEVALIN®) commercially available from IDEC Pharmaceuticals, Inc. (U.S. Pat. No. 5,736,137; 2B8 deposited with ATCC under accession no. HB11388 on Jun. 22, 1993); murine IgG2a “B1,” also called “Tositumomab,” optionally labelled with ¹³¹I to generate the “131I-B1” or “iodine I131 tositumomab” antibody (BEXXAR™, GlaxoSmithKline, see, also, U.S. Pat. No. 5,595,721); murine monoclonal antibody “1F5” (Press et al. Blood 69(2):584-591 (1987) and variants thereof including “framework patched” or humanized 1F5 (WO 2003/002607, Leung, S.; ATCC deposit HB-96450); murine 2H7 and chimeric 2H7 antibody (U.S. Pat. No. 5,677,180); a humanized 2H7 (WO 2004/056312 (Lowman et al.) and as set forth below); HuMAX-CD20™ a fully human antibody (Genmab, Denmark; see, for example, Glennie and van de Winkel, Drug Discovery Today 8: 503-510 (2003) and Cragg et al., Blood 101: 1045-1052 (2003)); the human monoclonal antibodies set forth in WO 2004/035607 (Teeling et al.); the antibodies having complex N-glycoside-linked sugar chains bound to the Fc region described in US 2004/0093621 (shitara et al.); CD20 binding molecules such as the AME series of antibodies, e.g., AME-133™ antibodies as set forth in WO 2004/103404 (Watkins et al., Applied Molecular Evolution); A20 antibody or variants thereof such as chimeric or humanized A20 antibody (cA20, IMMU-106a.k.a. hA20, respectively (US 2003/0219433, US 2005/0025764; Immunomedics); and monoclonal antibodies L27, G28-2, 93-1B3, B-C1 or NU-B2 available from the International Leukocyte Typing Workshop (Valentine et al., In: Leukocyte Typing III (McMichael, Ed., p. 440, Oxford University Press (1987)). The preferred CD20 antibodies herein are humanized, chimeric, or human CD20 antibodies, more preferably, a humanized 2H7 antibody, rituximab, chimeric or humanized A20 antibody (Immunomedics), and HuMAX-CD20™ human CD20 antibody (Genmab).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

### Compositions and Methods of the Invention

The invention provides pharmaceutical compositions for subcutaneous administration of a macromolecule, such as a protein, comprising 2% to 30% HP-Beta cyclodextrin, HP-Gamma cyclodextrin, or SBE cyclodextrin. In some embodiments, the invention provides a pharmaceutical formulation for subcutaneous administration of an antibody comprising an antibody at a concentration range of 10 mg/ml to 200 mg/ml, and 2% to 30% HP-Beta cyclodextrin, HP-Gamma cyclodextrin, or SBE cyclodextrin. In certain embodiments, the antibody concentration range is from 30-150 mg/ml. In further embodiments, the antibody concentration range is from 100-150 mg/ml. In certain embodiments, the pharmaceutical formulation comprises HP-Beta cyclodextrin at a concentration of 5% to 30%. In certain embodiments, the pharmaceutical formulation comprises HP-Gamma cyclodextrin at a concentration of 5% to 20%. In certain embodiments, the pharmaceutical formulation further comprises arginine succinate at a concentration of 50 mM to 200 mM. In certain embodiments, the pharmaceutical formulation comprises SBE cyclodextrin at a concentration of 2% to 9%. In certain embodiments, the pharmaceutical formulation comprises an antibody at a concentration of about 100 mg/ml and HP-Beta cyclodextrin at a concentration of 15% to 30%. In certain embodiments, the pharmaceutical formulation comprises an antibody at a concentration of about 150 mg/ml and HP-Beta cyclodextrin at a concentration of about 30%. In certain embodiments, the pharmaceutical formulation comprises an antibody at a concentration of about 150 mg/ml and HP-Gamma cyclodextrin at a concentration of about 10%. In certain embodiments, the pharmaceutical formulation further comprises arginine succinate at a concentration of 50 mM to 200 mM. In a specific embodiment, the pharmaceutical formulation comprises a humanized 2H7 antibody at a concentration range of 100 mg/ml to 150 mg/ml, HP-Gamma cyclodextrin at a concentration of 15% to 30%, and arginine succinate at a concentration of 50 mM to 100 mM. In further embodiments, the pharmaceutical composition further comprises 30 mM sodium acetate; 5% trehalose dihydrate; and 0.03% Polysorbate 20, at pH 5.3.

In various embodiments, the invention provides pharmaceutical compositions comprising humanized 2H7 antibodies (also referred to herein as hu2H7). In specific embodiments, the humanized 2H7 antibody is an antibody listed in Table 1.

TABLE 1
Humanized anti-CD20 Antibody and Variants Thereof

| | V_{L} | V_{H} | Full L chain | Full H chain |
|---|---|---|---|---|
| 2H7 Variant | SEQ ID NO. | SEQ ID NO. | SEQ ID NO. | SEQ ID NO. |
| A | 1 | 2 | 6 | 7 |
| B | 1 | 2 | 6 | 8 |
| C | 3 | 4 | 9 | 10 |
| D | 3 | 4 | 9 | 11 |
| F | 3 | 4 | 9 | 12 |
| G | 3 | 4 | 9 | 13 |
| H | 3 | 5 | 9 | 14 |
| I | 1 | 2 | 6 | 15 |

Each of antibody variants A, B and I of Table 1 comprises the light chain variable sequence (V_{L}):

(SEQ ID NO: 1)
DIQMTQSPSSLSASVGDRVTITCRASSSVSYMHWYQQKPGKAPKPLIY
APSNLASGVPSRFSGSGSGTDFTLTISSLQPEDFATYYCQQWSFNPPT
FGQGTKVEIKR;

and
the heavy chain variable sequence (V_{H}):

(SEQ ID NO: 2)
EVQLVESGGGLVQPGGSLRLSCAASGYTFTSYNMHWVRQAPGKGLEWV
GAIYPGNGDTSYNQKFKGRFTISVDKSKNTLYLQMNSLRAEDTAVYYC
ARVVYYSNSYWYFDVWGQGTLVTVSS.

Each of antibody variants C, D, F and G of Table 1 comprises the light chain variable sequence (V_{L}):

(SEQ ID NO: 3)
DIQMTQSPSSLSASVGDRVTITCRASSSVSYLHWYQQKPGKAPKPLIY
APSNLASGVPSRFSGSGSGTDFTLTISSLQPEDFATYYCQQWAFNPPT
FGQGTKVEIKR,

and
the heavy chain variable sequence (V_{H}):

(SEQ ID NO: 4)
EVQLVESGGGLVQPGGSLRLSCAASGYTFTSYNMHWVRQAPGKGLEWV
GAIYPGNGATSYNQKFKGRFTISVDKSKNTLYLQMNSLRAEDTAVYYC
ARVVYYSASYWYFDVWGQGTLVTVSS.

The antibody variant H of Table 1 comprises the light chain variable sequence (V_{L}) of SEQ ID NO:3 (above) and the heavy chain variable sequence (V_{H}):

(SEQ ID NO. 5)
EVQLVESGGGLVQPGGSLRLSCAASGYTFTSYNMHWVRQAPGKGLEWV
GAIYPGNGATSYNQKFKGRFTISVDKSKNTLYLQMNSLRAEDTAVYYC
ARVVYYSYRYWYFDVWGQGTLVTVSS.

Each of antibody variants A, B and I of Table 1 comprises the full length light chain sequence:

(SEQ ID NO: 6)
DIQMTQSPSSLSASVGDRVTITCRASSSVSYMHWYQQKPGKAPKPLIY
APSNLASGVPSRFSGSGSGTDFTLTISSLQPEDFATYYCQQWSFNPPT
FGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYA
CEVTHQGLSSPVTKSFNRGEC.

Variant A of Table 1 comprises the full length heavy chain sequence:

(SEQ ID NO: 7)
EVQLVESGGGLVQPGGSLRLSCAASGYTFTSYNMHWVRQAPGKGLEWV
GAIYPGNGDTSYNQKFKGRFTISVDKSKNTLYLQMNSLRAEDTAVYYC
ARVVYYSNSYWYFDVWGQGTLVTVSSASTKGPSVFPLAPSSKSTSGGT
AALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSV
MHEALHNHYTQKSLSLSPGK.

Variant B of Table 1 comprises the full length heavy chain sequence:

(SEQ ID NO: 8)
EVQLVESGGGLVQPGGSLRLSCAASGYTFTSYNMHWVRQAPGKGLEWV
GAIYPGNGDTSYNQKFKGRFTISVDKSKNTLYLQMNSLRAEDTAVYYC
ARVVYYSNSYWYFDVWGQGTLVTVSSASTKGPSVFPLAPSSKSTSGGT
AALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVE
VHNAKTKPREEQYNATYRVVSVLTVLHQDWLNGKEYKCKVSNKALPAP
IAATISKAKGQPREPQVYTLPPSREEMTKNQVSLTCLVKGFYPSDIAV
EWESNGQPENNYKTTPPVLDSDGSFFLYSKLTVDKSRWQQGNVFSCSV
MHEALHNHYTQKSLSLSPGK.

Variant I of Table 1 comprises the full length heavy chain sequence:

(SEQ ID NO: 15)
EVQLVESGGGLVQPGGSLRLSCAASGYTFTSYNMHWVRQAPGKGLEWV
GAIYPGNGDTSYNQKFKGRFTISVDKSKNTLYLQMNSLRAEDTAVYYC
ARVVYYSNSYWYFDVWGQGTLVTVSSASTKGPSVFPLAPSSKSTSGGT
AALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVE
VHNAKTKPREEQYNATYRVVSVLTVLHQDWLNGKEYKCKVSNAALPAP
IAATISKAKGQPREPQVYTLPPSREEMTKNQVSLTCLVKGFYPSDIAV
EWESNGQPENNYKTTPPVLDSDGSFFLYSKLTVDKSRWQQGNVFSCSV
MHEALHNHYTQKSLSLSPGK.

Each of antibody variants C, D, F, G and H of Table 1 comprises the full length light chain sequence:

(SEQ ID NO: 9)
DIQMTQSPSSLSASVGDRVTITCRASSSVSYLHWYQQKPGKAPKPLIY
APSNLASGVPSRFSGSGSGTDFTLTISSLQPEDFATYYCQQWAFNPPT
FGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYA
CEVTHQGLSSPVTKSFNRGEC.

Variant C of Table 1 comprises the full length heavy chain sequence:

(SEQ ID NO: 10)
EVQLVESGGGLVQPGGSLRLSCAASGYTFTSYNMHWVRQAPGKGLEWV
GAIYPGNGATSYNQKFKGRFTISVDKSKNTLYLQMNSLRAEDTAVYYC
ARVVYYSASYWYFDVWGQGTLVTVSSASTKGPSVFPLAPSSKSTSGGT
AALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVE
VHNAKTKPREEQYNATYRVVSVLTVLHQDWLNGKEYKCKVSNKALPAP
IAATISKAKGQPREPQVYTLPPSREEMTKNQVSLTCLVKGFYPSDIAV
EWESNGQPENNYKTTPPVLDSDGSFFLYSKLTVDKSRWQQGNVFSCSV
MHEALHNHYTQKSLSLSPGK.

Variant D of Table 1 comprises the full length heavy chain sequence:

(SEQ ID NO: 11)
EVQLVESGGGLVQPGGSLRLSCAASGYTFTSYNMHWVRQAPGKGLEWV
GAIYPGNGATSYNQKFKGRFTISVDKSKNTLYLQMNSLRAEDTAVYYC
ARVVYYSASYWYFDVWGQGTLVTVSSASTKGPSVFPLAPSSKSTSGGT
AALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVE
VHNAKTKPREEQYNATYRVVSVLTVLHQDWLNGKEYKCAVSNKALPAP
IEATISKAKGQPREPQVYTLPPSREEMTKNQVSLTCLVKGFYPSDIAV
EWESNGQPENNYKTTPPVLDSDGSFFLYSKLTVDKSRWQQGNVFSCSV
MHEALHNHYTQKSLSLSPGK.

Variant F of Table 1 comprises the full length heavy chain sequence:

(SEQ ID NO: 12)
EVQLVESGGGLVQPGGSLRLSCAASGYTFTSYNMHWVRQAPGKGLEWV
GAIYPGNGATSYNQKFKGRFTISVDKSKNTLYLQMNSLRAEDTAVYYC
ARVVYYSASYWYFDVWGQGTLVTVSSASTKGPSVFPLAPSSKSTSGGT
AALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVE
VHNAKTKPREEQYNATYRVVSVLTVLHQDWLNGKEYKCKVSNAALPAP
IAATISKAKGQPREPQVYTLPPSREEMTKNQVSLTCLVKGFYPSDIAV
EWESNGQPENNYKTTPPVLDSDGSFFLYSKLTVDKSRWQQGNVFSCSV
MHEALHNHYTQKSLSLSPGK.

Variant G of Table 1 comprises the full length heavy chain sequence:

(SEQ ID NO: 13)
EVQLVESGGGLVQPGGSLRLSCAASGYTFTSYNMHWVRQAPGKGLEWV
GAIYPGNGATSYNQKFKGRFTISVDKSKNTLYLQMNSLRAEDTAVYYC
ARVVYYSASYWYFDVWGQGTLVTVSSASTKGPSVFPLAPSSKSTSGGT
AALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVE
VHNAKTKPREEQYNATYRVVSVLTVLHQDWLNGKEYKCKVSNAALPAP
IAATISKAKGQPREPQVYTLPPSREEMTKNQVSLTCLVKGFYPSDIAV
EWESNGQPENNYKTTPPVLDSDGSFFLYSKLTVDKSRWQQGNVFSCSV
MHEALHWHYTQKSLSLSPGK.

Variant H of Table 1 comprises the full length heavy chain sequence:

(SEQ ID NO 14)
EVQLVESGGGLVQPGGSLRLSCAASGYTFTSYNMHWVRQAPGKGLEWV
GAIYPGNGATSYNQKFKGRFTISVDKSKNTLYLQMNSLRAEDTAVYYC
ARVVYYSYRYWYFDVWGQGTLVTVSSASTKGPSVFPLAPSSKSTSGGT
AALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVE
VHNAKTKPREEQYNATYRVVSVLTVLHQDWLNGKEYKCKVSNAALPAP
IAATISKAKGQPREPQVYTLPPSREEMTKNQVSLTCLVKGFYPSDIAV
EWESNGQPENNYKTTPPVLDSDGSFFLYSKLTVDKSRWQQGNVFSCSV
MHEALHNHYTQKSLSLSPGK.

In certain embodiments, the humanized 2H7 antibody of the invention further comprises amino acid alterations in the IgG Fc and exhibits increased binding affinity for human FcRn over an antibody having wild-type IgG Fc, by at least 60 fold, at least 70 fold, at least 80 fold, more preferably at least 100 fold, preferably at least 125 fold, even more preferably at least 150 fold to about 170 fold.

The N-glycosylation site in IgG is at Asn297 in the CH2 domain. Humanized 2H7 antibody compositions of the present invention include compositions of any of the preceding humanized 2H7 antibodies having a Fc region, wherein about 80-100% (and preferably about 90-99%) of the antibody in the composition comprises a mature core carbohydrate structure which lacks fucose, attached to the Fc region of the glycoprotein. Such compositions were demonstrated herein to exhibit a surprising improvement in binding to FcγRIIIA(F158), which is not as effective as FcγRIIIA (V158) in interacting with human IgG. FcγRIIIA (F158) is more common than FcγRIIIA (V158) in normal, healthy African Americans and Caucasians. See Lehrnbecher et al. Blood 94:4220 (1999). Historically, antibodies produced in Chinese Hamster Ovary Cells (CHO), one of the most commonly used industrial hosts, contain about 2 to 6% in the population that are nonfucosylated. YB2/0 and Lec13, however, can produce antibodies with 78 to 98% nonfucosylated species. Shinkawa et al. J. Bio. Chem. 278 (5), 3466-347 (2003), reported that antibodies produced in YB2/0 and Lec13 cells, which have less FUT8 activity, show significantly increased ADCC activity in vitro. The production of antibodies with reduced fucose content are also described in e.g., Li et al. (GlycoFi) “Optimization of humanized IgGs in glycoengineered Pichia pastoris” in Nature Biology online publication 22 Jan. 2006; Niwa R. et al. Cancer Res. 64(6):2127-2133 (2004); US 2003/0157108 (Presta); U.S. Pat. No. 6,602,684 and US 2003/0175884 (Glycart Biotechnology); US 2004/0093621, US 2004/0110704, US 2004/0132140 (all of Kyowa Hakko Kogyo).

The formulation herein may also contain more than one active compound as necessary for the particular indication being treated, preferably those with complementary activities that do not adversely affect each other. For example, it may be desirable to further provide a cytotoxic agent, chemotherapeutic agent, cytokine or immunosuppressive agent (e.g. one which acts on T cells, such as cyclosporin or an antibody that binds T cells, e.g. one which binds LFA-1). The effective amount of such other agents depends on the amount of antibody present in the formulation, the type of disease or disorder or treatment, and other factors discussed above. These are generally used in the same dosages and with administration routes as described herein or about from 1 to 99% of the heretofore employed dosages.

The formulations to be used for in vivo administration must be sterile. This is readily accomplished by filtration through sterile filters.

### Antibody Production

Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g., by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (C_{H} and C_{L}) sequences for the homologous murine sequences (U.S. Pat. No. 4,816,567; and Morrison, et al., Proc. Natl. Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

The humanized antibody may be an antibody fragment, such as a Fab, which is optionally conjugated with one or more cytotoxic agent(s) in order to generate an immunoconjugate. Alternatively, the humanized antibody may be an full length antibody, such as an full length IgG1 antibody.

Human Antibodies and Phage Display Methodology

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); 5,545,807; and WO 97/17852.

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

Antibody Fragments

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

Other Amino Acid Sequence Modifications

Amino acid sequence modification(s) of the CD20 binding antibodies described herein are contemplated. For example, it may be desirable to improve the binding affinity and/or other biological properties of the antibody. Amino acid sequence variants of the anti-CD20 antibody are prepared by introducing appropriate nucleotide changes into the anti-CD20 antibody nucleic acid, or by peptide synthesis. Such modifications include, for example, deletions from, and/or insertions into and/or substitutions of, residues within the amino acid sequences of the anti-CD20 antibody. Any combination of deletion, insertion, and substitution is made to arrive at the final construct, provided that the final construct possesses the desired characteristics. The amino acid changes also may alter post-translational processes of the anti-CD20 antibody, such as changing the number or position of glycosylation sites.

A useful method for identification of certain residues or regions of the anti-CD20 antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with CD20 antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed anti-CD20 antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an anti-CD20 antibody with an N-terminal methionyl residue or the antibody fused to a cytotoxic polypeptide. Other insertional variants of the anti-CD20 antibody molecule include the fusion to the N- or C-terminus of the anti-CD20 antibody to an enzyme (e.g. for ADEPT) or a polypeptide which increases the serum half-life of the antibody.

Another type of variant is an amino acid substitution variant. These variants have at least one amino acid residue in the anti-CD20 antibody molecule replaced by a different residue. The sites of greatest interest for substitutional mutagenesis include the hypervariable regions, but FR alterations are also contemplated. Conservative substitutions are shown in the Table below under the heading of “preferred substitutions”. If such substitutions result in a change in biological activity, then more substantial changes, denominated “exemplary substitutions” in the Table, or as further described below in reference to amino acid classes, may be introduced and the products screened.

TABLE 2
Amino Acid Substitutions

| | Exemplary | Preferred |
|---|---|---|
| Original Residue | Substitutions | Substitutions |
| Ala (A) | val; leu; ile | Val |
| Arg (R) | lys; gln; asn | Lys |
| Asn (N) | gln; his; asp, lys; arg | Gln |
| Asp (D) | glu; asn | Glu |
| Cys (C) | ser; ala | Ser |
| Gln (Q) | asn; glu | Asn |
| Glu (E) | asp; gln | Asp |
| Gly (G) | ala | Ala |
| His (H) | asn; gln; lys; arg | Arg |
| Ile (I) | leu; val; met; ala; phe; norleucine | Leu |
| Leu (L) | norleucine; ile; val; met; ala; phe | Ile |
| Lys (K) | arg; gln; asn | Arg |
| Met (M) | leu; phe; ile | Leu |
| Phe (F) | leu; val; ile; ala; tyr | Tyr |
| Pro (P) | ala | Ala |
| Ser (S) | thr | Thr |
| Thr (T) | ser | Ser |
| Trp (W) | tyr; phe | Tyr |
| Tyr (Y) | trp; phe; thr; ser | Phe |
| Val (V) | ile; leu; met; phe; ala; norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: tip, tyr, phe.

Non-conservative substitutions will entail exchanging a member of one of these classes for another class.

Any cysteine residue not involved in maintaining the proper conformation of the anti-CD20 antibody also may be substituted, generally with serine, to improve the oxidative stability of the molecule and prevent aberrant crosslinking Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and human CD20. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-CD20 antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-CD20 antibody.

It may be desirable to modify the antibody of the invention with respect to effector function, e.g. so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement mediated lysis and ADCC capabilities. See Stevenson et al. Anti-Cancer Drug Design 3:219-230 (1989).

### Therapeutic Uses

The disclosed methods and compositions comprising humanized 2H7CD20 binding antibodies of the invention are useful to treat a number of malignant and non-malignant diseases including CD20 positive B cell cancers such as B cell lymphomas and leukemia, and autoimmune diseases. Stem cells (B-cell progenitors) in bone marrow lack the CD20 antigen, allowing healthy B-cells to regenerate after treatment and return to normal levels within several months.

CD20 positive B cell cancers are those comprising abnormal proliferation of B cells that express CD20 on the cell surface. The CD20 positive B cell neoplasms include CD20-positive Hodgkin's disease including lymphocyte predominant Hodgkin's disease (LPHD); non-Hodgkin's lymphoma (NHL); follicular center cell (FCC) lymphomas; acute lymphocytic leukemia (ALL); chronic lymphocytic leukemia (CLL); Hairy cell leukemia.

The term “non-Hodgkin's lymphoma” or “NHL”, as used herein, refers to a cancer of the lymphatic system other than Hodgkin's lymphomas. Hodgkin's lymphomas can generally be distinguished from non-Hodgkin's lymphomas by the presence of Reed-Sternberg cells in Hodgkin's lymphomas and the absence of said cells in non-Hodgkin's lymphomas. Examples of non-Hodgkin's lymphomas encompassed by the term as used herein include any that would be identified as such by one skilled in the art (e.g., an oncologist or pathologist) in accordance with classification schemes known in the art, such as the Revised European-American Lymphoma (REAL) scheme as described in Color Atlas of Clinical Hematology (3rd edition), A. Victor Hoffbrand and John E. Pettit (eds.) (Harcourt Publishers Ltd., 2000). See, in particular, the lists in FIGS. 11.57, 11.58 and 11.59. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

In specific embodiments, pharmaceutical compositions comprising humanized CD20 binding antibodies and functional fragments thereof are used to treat non-Hodgkin's lymphoma (NHL), lymphocyte predominant Hodgkin's disease (LPHD), small lymphocytic lymphoma (SLL), and chronic lymphocytic leukemia (CLL), including relapses of these conditions.

Indolent lymphoma is a slow-growing, incurable disease in which the average patient survives between six and 10 years following numerous periods of remission and relapse. In one embodiment, the humanized CD20 binding antibodies or functional fragments thereof are used to treat indolent NHL including relapsed indolent NHL and rituximab-refractory indolent NHL. The relapsed indolent NHL patients can be Rituximab responders who have previously received one course of Rituximab and have responded for >6 months.

The present humanized 2H7 antibodies or functional fragments thereof are useful as a single-agent treatment (monotherapy) in, e.g., for relapsed or refractory low-grade or follicular, CD20-positive, B-cell NHL, or can be administered to patients in conjunction with other drugs in a multi-drug regimen.

The humanized 2H7 antibodies or functional fragments of the invention can be used as front-line therapy. The invention also contemplates the use of these antibodies for the treatment of patients with CD20 positive B cell neoplasms that are nonresponsive or have an inadequate response to treatment with any one of the following drugs: rituximab (Genentech); ibritumomab tiuxetan (Zevalin™, Biogen Idec); tositumomab (Bexxar™, GlaxoSmithKline); HuMAX-CD20™ (GenMab); IMMU-106 (which is a humanized anti-CD20a.k.a. hA20 or 90Y-hLL2, Immunomedics); AME-133 (Applied Molecular Evolution/Eli Lilly); gentuzumab ozogamicin (Mylotarg™, a humanized anti-CD33 antibody, Wyeth/PDL); alemtuzumab (Campath™, an anti-CD52 antibody, Schering Plough/Genzyme); epratuzumab (IMMU-103™, a humanized anti-CD22 antibody, Immunomedics), or have relapsed after treatment with these drugs.

The invention further provides a method of treating CLL patients including those who have failed fludarabine therapy, with the humanized 2H7 antibodies of the invention.

An “autoimmune disease” herein is a disease or disorder arising from and directed against an individual's own tissues or a co-segregate or manifestation thereof or resulting condition therefrom. Examples of autoimmune diseases or disorders include, but are not limited to arthritis (rheumatoid arthritis such as acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, and ataxic sclerosis, inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), pyoderma gangrenosum, erythema nodosum, primary sclerosing cholangitis, episcleritis), respiratory distress syndrome, including adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, allergic conditions and responses, allergic reaction, eczema including allergic or atopic eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis (including large vessel vasculitis (including polymyalgia rheumatica and giant cell (Takayasu's) arteritis), medium vessel vasculitis (including Kawasaki's disease and polyarteritis nodosa/periarteritis nodosa), microscopic polyarteritis, CNS vasculitis, necrotizing, cutaneous, or hypersensitivity vasculitis, systemic necrotizing vasculitis, and ANCA-associated vasculitis, such as Churg-Strauss vasculitis or syndrome (CSS)), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, immune complex nephritis, antibody-mediated nephritis, neuromyelitis optica, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia such as idiopathic thrombocytopenic purpura (ITP) including chronic or acute ITP, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis (LIP), bronchiolitis obliterans (non-transplant) vs NSIP, Guillain-Barré syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, infertility due to antispermatozoan antibodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, lymphadenitis, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

In specific embodiments, pharmaceutical compositions comprising humanized 2H7 antibodies and functional fragments thereof are used to treat rheumatoid arthritis and juvenile rheumatoid arthritis, systemic lupus erythematosus (SLE) including lupus nephritis, Wegener's disease, inflammatory bowel disease, ulcerative colitis, idiopathic thrombocytopenic purpura (ITP), thrombotic throbocytopenic purpura (TTP), autoimmune thrombocytopenia, multiple sclerosis including relapsed remitting MS, psoriasis, IgA nephropathy, IgM polyneuropathies, myasthenia gravis, ANCA associated vasculitis, diabetes mellitus, Reynaud's syndrome, Sjogren's syndrome, Neuromyelitis Optica (NMO) and glomerulonephritis.

“Treating” or “treatment” or “alleviation” refers to therapeutic treatment wherein the object is to slow down (lessen) if not cure the targeted pathologic condition or disorder or prevent recurrence of the condition. A subject is successfully “treated” for an autoimmune disease or a CD20 positive B cell malignancy if, after receiving a therapeutic amount of a humanized CD20 binding antibody of the invention according to the methods of the present invention, the subject shows observable and/or measurable reduction in or absence of one or more signs and symptoms of the particular disease. For example, for cancer, significant reduction in the number of cancer cells or absence of the cancer cells; reduction in the tumor size; inhibition (i.e., slow to some extent and preferably stop) of tumor metastasis; inhibition, to some extent, of tumor growth; increase in length of remission, slowing down in the progression of the disease, and/or relief to some extent, one or more of the symptoms associated with the specific cancer; reduced morbidity and mortality, and improvement in quality of life issues. Reduction of the signs or symptoms of a disease may also be felt by the patient. Treatment can achieve a complete response, defined as disappearance of all signs of cancer, or a partial response, wherein the size of the tumor is decreased, preferably by more than 50 percent, more preferably by 75%. A patient is also considered treated if the patient experiences stable disease. In one criterion, the h2H7 antibodies of the invention achieve >95% peripheral blood B cell depletion and the B cells return to 25% of baseline. In preferred embodiments, treatment with the antibodies of the invention is effective to result in the cancer patients being progression-free in the cancer 4 months after treatment, preferably 6 months, more preferably one year, even more preferably 2 or more years post treatment. These parameters for assessing successful treatment and improvement in the disease are readily measurable by routine procedures familiar to a physician of appropriate skill in the art.

A “therapeutically effective amount” refers to an amount of an antibody or a drug effective to “treat” a disease or disorder in a subject. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See preceding definition of “treating”. In the case of an autoimmune disease, the therapeutically effective amount of the antibody or other drug is effective to reduce the signs and symptoms of the disease.

The parameters for assessing efficacy or success of treatment of the neoplasm will be known to the physician of skill in the appropriate disease. Generally, the physician of skill will look for reduction in the signs and symptoms of the specific disease. Parameters can include median time to disease progression, time in remission, stable disease.

The following references describe lymphomas and CLL, their diagnoses, treatment and standard medical procedures for measuring treatment efficacy. Canellos G P, Lister, T A, Sklar J L: The Lymphomas. W.B. Saunders Company, Philadelphia, 1998; van Besien K and Cabanillas, F: Clinical Manifestations, Staging and Treatment of Non-Hodgkin's Lymphoma, Chap. 70, pp 1293-1338, in: Hematology, Basic Principles and Practice, 3rd ed. Hoffman et al. (editors). Churchill Livingstone, Philadelphia, 2000; and Rai, K and Patel, D:Chronic Lymphocytic Leukemia, Chap. 72, pp 1350-1362, in: Hematology, Basic Principles and Practice, 3rd ed. Hoffman et al. (editors). Churchill Livingstone, Philadelphia, 2000.

The parameters for assessing efficacy or success of treatment of an autoimmune or autoimmune related disease will be known to the physician of skill in the appropriate disease. Generally, the physician of skill will look for reduction in the signs and symptoms of the specific disease. The following are by way of examples.

In one embodiment, pharmaceutical compositions comprising the humanized 2H7 antibodies are used to treat rheumatoid arthritis.

RA is a debilitating autoimmune disease that affects more than two million Americans and hinders the daily activities of sufferers. RA occurs when the body's own immune system inappropriately attacks joint tissue and causes chronic inflammation that destroys healthy tissue and damage within the joints. Symptoms include inflammation of the joints, swelling, stiffness, and pain. Additionally, since RA is a systemic disease, it can have effects in other tissues such as the lungs, eyes and bone marrow. There is no known cure. Treatments include a variety of steroidal and non-steroidal anti-inflammatory drugs, immunosuppressive agents, disease-modifying anti-rheumatic drugs (DMARDs), and biologics. However, many patients continue to have an inadequate response to treatment.

The antibodies can be used as first-line therapy in patients with early RA (i.e., methotrexate (MTX) naive) and as monotherapy, or in combination with or following, e.g., MTX or cyclophosphamide. Or, the antibodies can be used in treatment as second-line therapy for patients who were DMARD and/or MTX refractory, and as monotherapy or in combination with, e.g., MTX. The humanized CD20 binding antibodies are useful to prevent and control joint damage, delay structural damage, decrease pain associated with inflammation in RA, and generally reduce the signs and symptoms in moderate to severe RA. The RA patient can be treated with the humanized CD20 antibody prior to, after or together with treatment with other drugs used in treating RA (see combination therapy below). In one embodiment, patients who had previously failed disease-modifying antirheumatic drugs and/or had an inadequate response to methotrexate alone are treated with a humanized CD20 binding antibody of the invention. In one embodiment of this treatment, the patients are in a 17-day treatment regimen receiving humanized CD20 binding antibody alone (1 g i.v. infusions on days 1 and 15); CD20 binding antibody plus cyclophosphamide (750 mg i.v. infusion days 3 and 17); or CD20 binding antibody plus methotrexate.

Because the body produces tumor necrosis factor alpha (TNFα) during RA, TNFα inhibitors have been used for therapy of that disease. However, TNFα inhibitors such as Etanercept (ENBREL®), Infliximab (REMICADE®) and Adalimumab (HUMIRA™) can produce negative side effects such as infection, heart failure and demyelination. Therefore, in one embodiment, the humanized CD20 binding antibodies or biologically functional fragments thereof are useful, for example as first-line therapy, to treat RA patients to reduce the risk of these negative side effects experienced with TNFα inhibitor drugs or to treat patients considered to be prone to experience a toxicity, e.g. cardiac toxicity. The humanized CD20 binding antibodies or biologically functional fragments thereof are also useful in a method of treating a subject suffering from RA who has been treated with a TNFα-inhibitor but is nonresponsive, has an inadequate response to the TNFα-inhibitor (TNF-IR patients), or has a relapse of disease after some time of response, or determined to be one who is unlikely to respond to therapy with a TNFα-inhibitor. In one embodiment the TNF-IR are treated with a low dose such as below 100 mg, prior to treatment with a TNFα inhibitor.

One method of evaluating treatment efficacy in RA is based on American College of Rheumatology (ACR) criteria, which measures the percentage of improvement in tender and swollen joints, among other things. The RA patient can be scored at for example, ACR 20 (20 percent improvement) compared with no antibody treatment (e.g., baseline before treatment) or treatment with placebo. Other ways of evaluating the efficacy of antibody treatment include X-ray scoring such as the Sharp X-ray score used to score structural damage such as bone erosion and joint space narrowing. Patients can also be evaluated for the prevention of or improvement in disability based on Health Assessment Questionnaire [HAQ] score, AIMS score, SF-36 at time periods during or after treatment. The ACR 20 criteria may include 20% improvement in both tender (painful) joint count and swollen joint count plus a 20% improvement in at least 3 of 5 additional measures:
1. patient's pain assessment by visual analog scale (VAS),
2. patient's global assessment of disease activity (VAS),
3. physician's global assessment of disease activity (VAS),
4. patient's self-assessed disability measured by the Health Assessment Questionnaire, and
5. acute phase reactants, CRP or ESR.
   The ACR 50 and 70 are defined analogously. Preferably, the patient is administered an amount of a CD20 binding antibody of the invention effective to achieve at least a score of ACR 20, preferably at least ACR 30, more preferably at least ACR50, even more preferably at least ACR70, most preferably at least ACR 75 and higher.

Psoriatic arthritis has unique and distinct radiographic features. For psoriatic arthritis, joint erosion and joint space narrowing can be evaluated by the Sharp score as well. The humanized CD20 binding antibodies of the invention can be used to prevent the joint damage as well as reduce disease signs and symptoms of the disorder.

Yet another aspect of the invention is a method of treating SLE or lupus nephritis by administering to a subject suffering from the disorder, a pharmaceutical composition comprising a therapeutically effective amount of a humanized CD20 binding antibody of the invention. SLEDAI scores provide a numerical quantitation of disease activity. The SLEDAI is a weighted index of 24 clinical and laboratory parameters known to correlate with disease activity, with a numerical range of 0-103. see Bryan Gescuk & John Davis, “Novel therapeutic agent for systemic lupus erythematosus” in Current Opinion in Rheumatology 2002, 14:515-521. Other scoring methods include BILAG scoring. Antibodies to double-stranded DNA are believed to cause renal flares and other manifestations of lupus. Patients undergoing antibody treatment can be monitored for time to renal flare, which is defined as a significant, reproducible increase in serum creatinine, urine protein or blood in the urine. Alternatively or in addition, patients can be monitored for levels of antinuclear antibodies and antibodies to double-stranded DNA. Treatments for SLE include high-dose corticosteroids and/or cyclophosphamide (HDCC). Herein, a successful treatment of lupus would reduce flare i.e., reduce the severity and/or time to the next flare.

Spondyloarthropathies are a group of disorders of the joints, including ankylosing spondylitis, psoriatic arthritis and Crohn's disease. Treatment success can be determined by validated patient and physician global assessment measuring tools.

With regard to vasculitis, approximately 75% of the patients with systemic vasculitides have anti-neutrophil cytoplasmic antibody and cluster into one of three conditions affecting small/medium sized vessels: Wegener's granulomatosus (WG), microscopic polyangiitis (MPA) and Churg Strauss syndrome (CSS), collectively known as ANCA associated vasculitis (AAV).

Treatment efficacy for psoriasis is assessed by monitoring changes in clinical signs and symptoms of the disease including Physician's Global Assessment (PGA) changes and Psoriasis Area and Severity Index (PASI) scores, Psoriasis Symptom Assessment (PSA), compared with the baseline condition. The psoriasis patient treated with a humanized CD20 binding antibody of the invention such as hu2H7.v511 can be measured periodically throughout treatment on the Visual analog scale used to indicate the degree of itching experienced at specific time points.

Patients may experience an infusion reaction or infusion-related symptoms with their first infusion of a therapeutic antibody. These symptoms vary in severity and generally are reversible with medical intervention. These symptoms include but are not limited to, flu-like fever, chills/rigors, nausea, urticaria, headache, bronchospasm, angioedema. It would be desirable for the disease treatment methods of the present invention to minimize infusion reactions. To alleviate or minimize such adverse events, the patient may receive an initial conditioning or tolerizing dose(s) of the antibody followed by a therapeutically effective dose. The conditioning dose(s) will be lower than the therapeutically effective dose to condition the patient to tolerate higher dosages.

Dosing

Depending on the indication to be treated and factors relevant to the dosing that a physician of skill in the field would be familiar with, the antibodies of the invention will be administered at a dosage that is efficacious for the treatment of that indication while minimizing toxicity and side effects. The desired dosage may depend on the disease and disease severity, stage of the disease, level of B cell modulation desired, and other factors familiar to the physician of skill in the art.

For treatment of an autoimmune disease, it may be desirable to modulate the extent of B cell depletion depending on the disease and/or the severity of the condition in the individual patient, by adjusting the dosage of humanized 2H7 antibody. B cell depletion can but does not have to be complete. Or, total B cell depletion may be desired in initial treatment but in subsequent treatments, the dosage may be adjusted to achieve only partial depletion. In one embodiment, the B cell depletion is at least 20%, i.e., 80% or less of CD20 positive B cells remain as compared to the baseline level before treatment. In other embodiments, B cell depletion is 25%, 30%, 40%, 50%, 60%, 70% or greater. Preferably, the B cell depletion is sufficient to halt progression of the disease, more preferably to alleviate the signs and symptoms of the particular disease under treatment, even more preferably to cure the disease.

The antibodies of the invention can be administered at various dosing fequencies, e.g., weekly, biweekly, monthly, etc. In an example, the dosing frequency is one dose every six months, or two doses spaced across two weeks every six months. The volume of the antibody solution to be injected can range from about 01. to about 3 ml per injection, more preferably from about 0.5 ml to about 1.5 ml per injection. The total amount of humanized 2H7 antibody administered in one injection can be up to about 150 mg per injection. Multiple injections may be used in order to achieve a desired dose.

Patients having an autoimmune disease or a B cell malignancy for whom one or more current therapies were ineffective, poorly tolerated, or contraindicated can be treated using any of the dosing regimens of the present invention. For example, the invention contemplates the present treatment methods for RA patients who have had an inadequate response to tumor necrosis factor (TNF) inhibitor therapies or to disease-modifying anti-rheumatic drugs (DMARD) therapy.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Combination Therapy

In treating the B cell neoplasms described above, the patient can be treated with the humanized 2H7 antibodies of the present invention in conjunction with one or more therapeutic agents such as a chemotherapeutic agent in a multidrug regimen. The humanized 2H7 antibody can be administered concurrently, sequentially, or alternating with the chemotherapeutic agent, or after non-responsiveness with other therapy. Standard chemotherapy for lymphoma treatment may include cyclophosphamide, cytarabine, melphalan and mitoxantrone plus melphalan. CHOP is one of the most common chemotherapy regimens for treating Non-Hodgkin's lymphoma. The following are the drugs used in the CHOP regimen: cyclophosphamide (brand names cytoxan, neosar); adriamycin (doxorubicin/hydroxydoxorubicin); vincristine (Oncovin); and prednisolone (sometimes called Deltasone or Orasone). In particular embodiments, the CD20 binding antibody is administered to a patient in need thereof in combination with one or more of the following chemotherapeutic agents of doxorubicin, cyclophosphamide, vincristine and prednisolone. In a specific embodiment, a patient suffering from a lymphoma (such as a non-Hodgkin's lymphoma) is treated with a humanized 2H7 antibody of the present invention in conjunction with CHOP (cyclophosphamide, doxorubicin, vincristine and prednisone) therapy. In another embodiment, the cancer patient can be treated with a humanized 2H7CD20 binding antibody of the invention in combination with CVP (cyclophosphamide, vincristine, and prednisone) chemotherapy. In a specific embodiment, the patient suffering from CD20-positive NHL is administered humanized 2H7.v511 or v114 in conjunction with CVP, for example, every 3 weeks for 8 cycles. In a specific embodiment of the treatment of CLL, the hu2H7.v511 antibody is administered in conjunction with chemotherapy with one or both of fludarabine and cytoxan.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; TLK 286 (TELCYTA™); acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; bisphosphonates, such as clodronate; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)) and anthracyclines such as annamycin, AD 32, alcarubicin, daunorubicin, dexrazoxane, DX-52-1, epirubicin, GPX-100, idarubicin, KRN5500, menogaril, dynemicin, including dynemicin A, an esperamicin, neocarzinostatin chromophore and related chromoprotein enediyne antibiotic chromophores, aclacinomysins, actinomycin, authramycin, azaserine, bleomycins, cactinomycin, carabicin, caminomycin, carzinophilin, chromomycinis, dactinomycin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenisher such as folinic acid (leucovorin); aceglatone; anti-folate anti-neoplastic agents such as ALIMTA®, LY231514 pemetrexed, dihydrofolate reductase inhibitors such as methotrexate, anti-metabolites such as 5-fluorouracil (5-FU) and its prodrugs such as UFT, S-1 and capecitabine, and thymidylate synthase inhibitors and glycinamide ribonucleotide formyltransferase inhibitors such as raltitrexed (TOMUDEX®, TDX); inhibitors of dihydropyrimidine dehydrogenase such as eniluracil; aldophosphamide glycoside; aminolevulinic acid; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); cyclophosphamide; thiotepa; taxoids and taxanes, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; gemcitabine (GEMZAR®); 6-thioguanine; mercaptopurine; platinum; platinum analogs or platinum-based analogs such as cisplatin, oxaliplatin and carboplatin; vinblastine (VELBAN®); etoposide (VP-16); ifosfamide; mitoxantrone; vincristine (ONCOVIN®); vinca alkaloid; vinorelbine (NAVELBINE®); novantrone; edatrexate; daunomycin; aminopterin; xeloda; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

Additionally, the hu2H7 antibodies and functional fragments thereof can be used to treat a CD20 expressing B cell neoplasm (e.g, NHL) in conjunction with an anti-tumor angiogenesis agent such as a Vascular Endothelial Growth Factor (VEGF) antagonist. An “anti-angiogenesis agent” or “angiogenesis inhibitor” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. For example, an anti-angiogenesis agent is an antibody or other antagonist to an angiogenic agent as defined above, e.g., antibodies to VEGF, antibodies to VEGF receptors, small molecules that block VEGF receptor signaling (e.g., PTK787/ZK2284, SU6668). A “VEGF antagonist” refers to a molecule capable of neutralizing, blocking, inhibiting, abrogating, reducing or interfering with VEGF activities including its binding to one or more VEGF receptors. In one embodiment, a patient suffering from such a B cell neoplasm is treated with 2H7.v511 or 2H7.v114 in conjuction with Avastin® (bevacizumab; Genentech). The anti-VEGF antibody “bevacizumab (BV)”, also known as “rhuMAb VEGF” or “Avastin®”, is a recombinant humanized anti-VEGF monoclonal antibody generated according to Presta et al. Cancer Res. 57:4593-4599 (1997).

The hu2H7 antibodies and functional fragments thereof are useful in a method of treating a CD20 expressing B cell neoplasm in conjunction with a member of the TNF family of cytokines such as Apo-2 ligand (Apo2L) also referred to as TRAIL. The full length native sequence human Apo-2 ligand is a 281 amino acid long, Type II transmembrane protein of the tumor necrosis factor family of cytokines Soluble forms of the Apo-2 ligand, such as those comprising an extracellular domain (ECD) or portions thereof, have been found to have various activities, including apoptotic activity in mammalian cancer cells. Apo2L/TRAIL (described in WO 97/01633 and WO 97/25428) is a soluble human protein which is a fragment of the ECD, comprising amino acid 114-281 of the full length Apo-2L protein.

In treating the autoimmune diseases or autoimmune related conditions described above, the patient can be treated with one or more hu2H7 antibodies, in conjunction with a second therapeutic agent, such as an immunosuppressive agent, such as in a multi drug regimen. The hu2H7 antibody can be administered concurrently, sequentially or alternating with the immunosuppressive agent or upon non-responsiveness with other therapy. The immunosuppressive agent can be administered at the same or lesser dosages than as set forth in the art. The preferred adjunct immunosuppressive agent will depend on many factors, including the type of disorder being treated as well as the patient's history.

“Immunosuppressive agent” as used herein for adjunct therapy refers to substances that act to suppress or mask the immune system of a patient. Such agents would include substances that suppress cytokine production, down regulate or suppress self-antigen expression, or mask the MHC antigens. Examples of such agents include steroids such as glucocorticosteroids, e.g., prednisone, methylprednisolone, and dexamethasone; 2-amino-6-aryl-5-substituted pyrimidines (see U.S. Pat. No. 4,665,077), azathioprine (or cyclophosphamide, if there is an adverse reaction to azathioprine); bromocryptine; glutaraldehyde (which masks the MHC antigens, as described in U.S. Pat. No. 4,120,649); anti-idiotypic antibodies for MHC antigens and MHC fragments; cyclosporin A; cytokine or cytokine receptor antagonists including anti-interferon-γ, -β, or -α antibodies; anti-tumor necrosis factor-α antibodies; anti-tumor necrosis factor-β antibodies; anti-interleukin-2 antibodies and anti-IL-2 receptor antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; TGF-β; streptodornase; RNA or DNA from the host; FK506; RS-61443; deoxyspergualin; rapamycin; T-cell receptor (U.S. Pat. No. 5,114,721); T-cell receptor fragments (Offner et al., Science 251:430-432 (1991); WO 90/11294; and WO 91/01133); and T cell receptor antibodies (EP 340,109) such as T10B9.

For the treatment of rheumatoid arthritis, the patient can be treated with a CD20 binding antibody of the invention in conjunction with any one or more of the following drugs: DMARDS (disease-modifying anti-rheumatic drugs (e.g., methotrexate), NSAI or NSAID (non-steroidal anti-inflammatory drugs), immunosuppressants (e.g., azathioprine; mycophenolate mofetil (CellCept®; Roche)), analgesics, glucocorticosteroids, cyclophosphamide, HUMIRA™ (adalimumab; Abbott Laboratories), ARAVA® (leflunomide), REMICADE® (infliximab; Centocor Inc., of Malvern, Pa.), ENBREL® (etanercept; Immunex, Wash.), ACTEMRA® (tocilizumab; Roche, Switzerland), COX-2 inhibitors. DMARDs commonly used in RA are hydroxycloroquine, sulfasalazine, methotrexate, leflunomide, etanercept, infliximab, azathioprine, D-penicillamine, Gold (oral), Gold (intramuscular), minocycline, cyclosporine, Staphylococcal protein A immunoadsorption.

Adalimumab is a human monoclonal antibody that binds to TNFα. Infliximab is a chimeric mouse-human monoclonal antibody that binds to TNFα. It is an immune-suppressing drug prescribed to treat RA and Crohn's disease. Infliximab has been linked to a fatal reactions such as heart failure and infections including tuberculosis as well as demyelination resulting in MS. Actemra (tocilizumab) is a humanized anti-human interleukin-6 (IL-6) receptor.

Etanercept is an “immunoadhesin” fusion protein consisting of the extracellular ligand binding portion of the human 75 kD (p75) tumor necrosis factor receptor (TNFR) linked to the Fc portion of a human IgG1. Etanercept (ENBREL®) is an injectable drug approved in the US for therapy of active RA. Etanercept binds to TNFα and serves to remove most TNFα from joints and blood, thereby preventing TNFα from promoting inflammation and other symptoms of rheumatoid arthritis. The drug has been associated with negative side effects including serious infections and sepsis, nervous system disorders such as multiple sclerosis (MS). See, e.g., www.remicade-infliximab.com/pages/enbrel_embrel.html

For conventional treatment of RA, see, e.g., “Guidelines for the management of rheumatoid arthritis” Arthritis & Rheumatism 46(2): 328-346 (February, 2002). In a specific embodiment, the RA patient is treated with a hu2H7CD20 antibody of the invention in conjunction with methotrexate (MTX). An exemplary dosage of MTX is about 7.5-25 mg/kg/wk. MTX can be administered orally and subcutaneously.

In one example, patients also receive concomitant MTX (10-25 mg/week per oral (p.o.) or parenteral), together with a corticosteroid regimen consisting of methylprednisolone 100 mg i.v. 30 minutes prior to infusions of the CD20 antibody and prednisone 60 mg p.o. on Days 2-7, 30 mg p.o. Days 8-14, returning to baseline dose by Day 16. Patients may also receive folate (5 mg/week) given as either a single dose or as divided daily doses. Patients optionally continue to receive any background corticosteroid (10 mg/d prednisone or equivalent) throughout the treatment period.

For the treatment of ankylosing spondylitis, psoriatic arthritis and Crohn's disease, the patient can be treated with a CD20 binding antibody of the invention in conjunction with, for example, Remicade® (infliximab; from Centocor Inc., of Malvern, Pa.), ENBREL (etanercept; Immunex, Wash.).

Treatments for SLE include combination of the CD20 antibody with high-dose corticosteroids and/or cyclophosphamide (HDCC). Patients suffering from SLE, AAV and NMO can be treated with a 2H7 antibody of the invention in combination with any of the following: corticosteroids, NSAIDs, analgesics, COX-2 inhibitors, glucocorticosteriods, conventional DMARDS (e.g. methotexate, sulphasalazine, hydroxychloroquine, leflunomide), biologic DMARDs such as anti-Blys (e.g., belimumab), anti-IL6R e.g., tocilizumab; CTLA4-Ig (abatacept), (anti-CD22 e.g., epratuzumab), immunosuppressants (e.g., azathioprine; mycophenolate mofetil (CellCept®; Roche)), and cytotoxic agents (e.g., cyclophosphamide).

For the treatment of psoriasis, patients can be administered a humanized 2H7 antibody in conjunction with topical treatments, such as topical steroids, anthralin, calcipotriene, clobetasol, and tazarotene, or with methotrexate, retinoids, cyclosporine, PUVA and UVB therapies. In one embodiment, the psoriasis patient is treated with a humanized 2H7 antibody sequentially or concurrently with cyclosporine.

To minimize toxicity, the traditional systemic therapies can be administered in rotational, sequential, combinatorial, or intermittent treatment regimens, or lower dosage combination regimens with the hu2H7CD20 binding antibody compositions at the present dosages.

### Articles of Manufacture and Kits

Another embodiment of the invention is an article of manufacture comprising a formulation of the invention useful for the treatment of autoimmune diseases and related conditions and CD20 positive cancers such as non-Hodgkin's lymphoma. The article of manufacture comprises a container and a label or package insert on or associated with the container. Suitable containers include, for example, bottles, vials, syringes, etc. The containers may be formed from a variety of materials such as glass or plastic. At least one active agent in the formulation or composition is a hu2H7 antibody of the invention, the antibody being present in the container such as a syringe, at an amount to deliver the dosage described above under dosing. The concentration of the hu2H7 will be in the range of 10 mg/ml to 200 mg/ml, can be 30-150 mg/ml or 100-150 mg/ml. The label or package insert indicates that the composition is used for treating the particular condition. The label or package insert will further comprise instructions for administering the antibody composition to the patient.

Package insert refers to instructions customarily included in commercial packages of therapeutic products, that contain information about the indications, usage, dosage, administration, contraindications and/or warnings concerning the use of such therapeutic products. In one embodiment, the package insert indicates that the composition is used for treating non-Hodgkins' lymphoma.

Additionally, the article of manufacture may further comprise a second container comprising a pharmaceutically-acceptable buffer, such as water of injection (WFI), bacteriostatic water for injection (BWFI), phosphate-buffered saline, Ringer's solution, sodium chloride (0.9%) and dextrose solution. It may further include other materials desirable from a commercial and user standpoint, including other buffers, diluents, filters, needles, and syringes.

### EXPERIMENTAL EXAMPLES

### Example 1

### Initial Subcutaneous Formulation for rhuMab 2H7

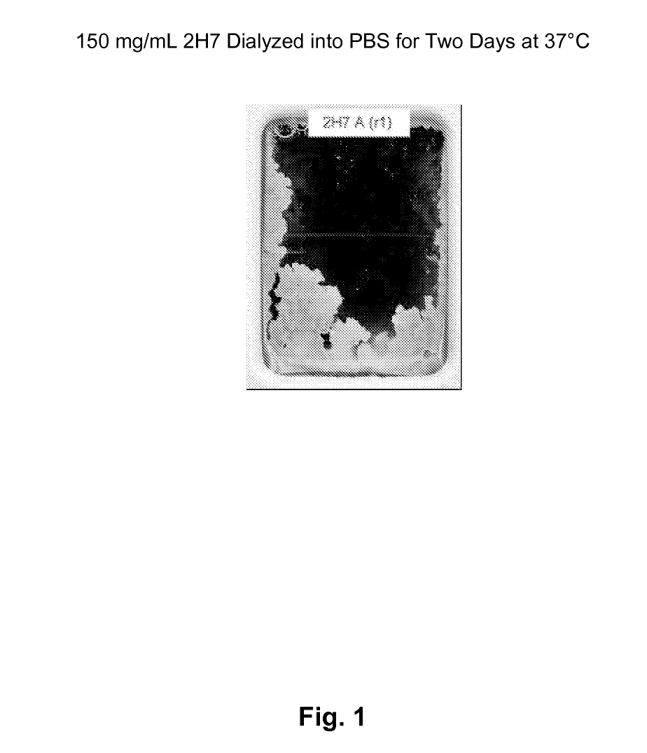

A high concentration subcutaneous formulation (150 mg/mL) was developed for rhuMAb 2H7. This formulation comprises 150 mg/ml 2H7, 30 mM sodium acetate; 7% trehalose dihydrate; 0.03% Polysorbate 20, at pH 5.3. This formulation is stable long term in the final vial storage under the recommended conditions. Administration of this material by subcutaneous injections in cynomolugus monkeys resulted in severe inflammation at the injection site and low bioavailability (≈30%). Mild to moderate macrophage infiltrate in the subcutaneous layer was observed in these animals. The cause of the irritation was attributed to foreign body material (i.e., 2H7 test material). Testing of this formulation under conditions that simulated what the product was exposed to at the injection site confirmed that the protein was significantly aggregated under physiological conditions (FIG. 1) corroborating the inflammation results observed in cynomologus monkeys. The observed precipitation may be consistent with a salting out effect subsequent to a pH shift.

### Example 2

### In Vitro Dialysis Method for Testing Macromolecule Aggregation Under the Physiologic Conditions of Subcutaneous Injection

An in vitro dialysis method was developed to test the ability of different excipients to reduce 2H7 aggregation under the physiologic conditions encountered during subcutaneous injection. A modified PBS solution (the “release medium”) was developed for this model to simulate the interstitial fluid. This in vitro system was used to evaluate the effect of sugars, polymers, surfactants, and amino acids in retarding 2H7 aggregation. Candidate formulations that showed improved product release in vitro were then tested in vivo (rat subcutaneous model; see Example 3) to determine if this improvement corresponded to decreased inflammation in vivo.

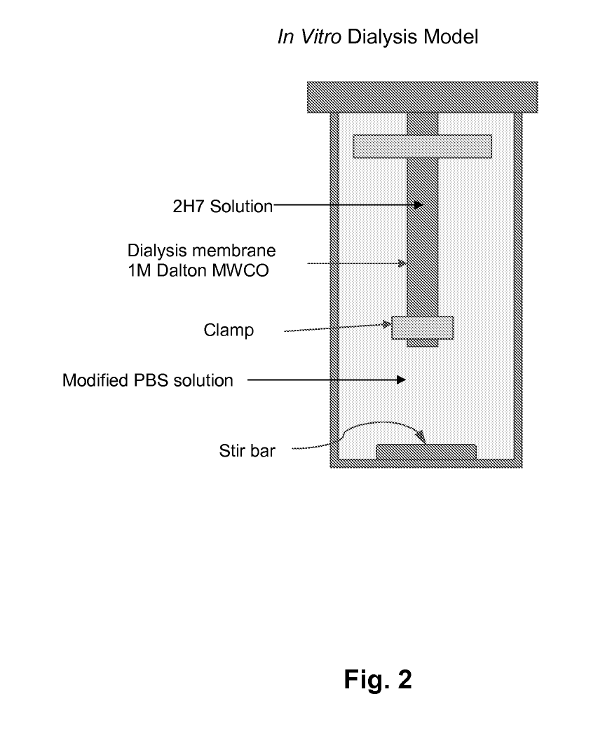

The set-up of the in vitro dialysis model is shown in FIG. 2. 250 ml glass jars were filled with 220 ml release medium (167 mM Sodium, 140 mM Chloride, 17 mM Phosphate, 4 mM Potassium) at 37° C. 6 cm lengths of dialysis tubing (Spectra Por 1 Million Molecular Weight Cut Off (MWCO) PVDF Dialysis tubing 12 mm diameter) were soaked in purified water. One end of the dialysis tubing was clamped, and the tubing was filled with approximately 1 ml of test sample (2H7 with test excipient). Excess air was removed, and the opposite end of the tubing was clamped to the seal of the jar. The filled bag was added to the 250 mL glass jar containing the release medium, and the jar was placed at 37° C. with constant stirring. 500 μl samples of the release medium were removed after 2.5, 6, 12, 24, 33 and 48 hours. The turbidity of the samples and the amount of protein present in the release medium were measured by UV photometric scan. In addition, the release medium and the solution inside the dialysis tubing were visually inspected for precipitation.

A test excipient was considered to be acceptable in the in vitro aggregations study if:
The cumulative release of 2H7 with the test excipient was greater than the negative control (original 2H7 formulation-150 mg/ml 2H7, 30 mM Sodium acetate; 7% trehalose Dihydrate; 0.03% Polysorbate 20, at pH 5.3) indicating improved 2H7 characteristics.
The positive control (rhuMAb anti-CD11a, also known as Raptiva™, a humanized anti-CD11a antibody administered subcutaneously) showed no precipitation and greater release than the negative control.
The precipitation of 2H7 was reduced or eliminated.
The turbidity of the release medium was reduced.

Candidates that met the acceptance criteria were then tested in the in vivo rat model to determine if retarding aggregation in vitro correlated to decreased inflammation in vivo.

### In Vitro Results:

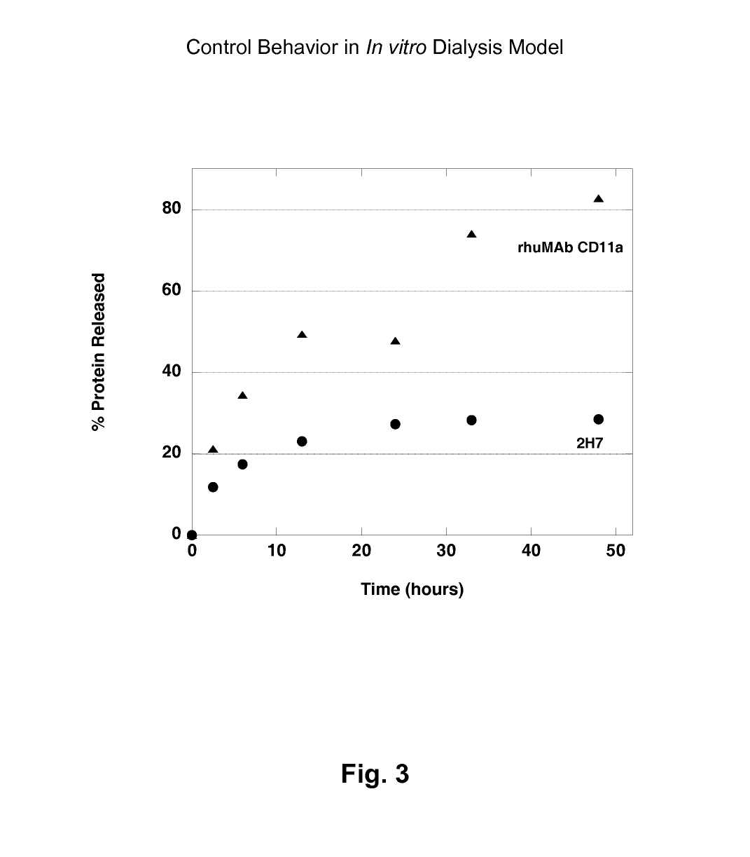

The typical release profile of the study controls in the in vitro dialysis method is shown in FIG. 3. The controls for this model were chosen to bracket release of a protein that did not readily aggregate (rhuMAb CD11a) and a release of protein that typically aggregated (original 2H7) under physiologic conditions. The area between the two release curves measures the relative ability of test excipients to retard aggregation relative to the controls.

The cumulative release of the original 2H7 formulation is low (<30%). Increased turbidity of the release medium was observed as 2H7 was released from the dialysis bag into the release medium, indicating that the material was aggregating in that environment. Extensive flocculation inside the dialysis bag was observed within 24 hours and corresponded to a dramatic decrease in 2H7 concentration from 150 mg/mL at the start of the study to 4 to 5 mg/mL by the end of the 48-hour study. All of these observations indicate that 2H7 readily aggregates under physiologic conditions. This behavior is not seen when the 2H7 original formulation is stored in a glass vial at 37° C. In contrast, rhuMAb CD11a is quickly released from the dialysis bag into the release medium. The release medium remained clear throughout the study and no flocculation was observed inside the dialysis bag, indicating that rhuMAb CD11a does not aggregate under physiologic conditions and is relevant as a control for this model. Table 3 summarizes the percentage protein released, release medium turbidity and presence of flocculation.

TABLE 3

| | | | Turbidity of | |
|---|---|---|---|---|
| | | | Release | Flocculation |
| | Time | % Cumulative | Medium | inside dialysis |
| Control | (hours) | Protein Released | OD 350 nm | bag |
| rhuMAb | 0 | 0 | 0.001 | No |
| CD11a | 48 | 83 | 0.03 | No |
| 2H7 Original | 0 | 0 | 0.02 | No |
| | 48 | 28 | 0.37 | Yes |

### Example 3

### In Vivo Rat Subcutaneous Model for Testing Macromolecule Aggregation

The rat subcutaneous model is a relevant model based on the similarity in character of the subcutaneous inflammation. The inflammatory response of rats receiving the original 2H7 formulation was consistent with the inflammatory response observed in the cynomologus monkeys (see Example 1). Immuno-histochemistry staining for human immunoglobulin was positive in sections of rat skin injected with 2H7, indicating the presence or persistence of the antibody in the areas of inflammation which supports the theory that precipitation of the test article caused inflammation at the injection site.

The in vivo rat screening assay was carried out as follows:

Each test or control formulation (0.25 ml) was administered subcutaneously. The animals were necropsied at 72 hours post dose. Skin sections at the injection sites were transected and fixed in formalin, and the effect of the test excipient on lowering inflammation was determined by histology. An inflammation score was assigned to the histology sections as follows:
+/−: minimum/slight inflammation
1: mild inflammation
2: moderate
3: severe

The presence of granuloma was determined by pathology. Tissue from the injection site was sectioned, stained and viewed under a light microscope for the presence or absence of granuloma.

The acceptance criteria for the in vivo rat model were: (1) comparable inflammation to rhuMAb CD11 a (negative control), and (2) absence of granuloma at injection site.

### Example 4

### Ability of Surfactants and Other Additives to Decrease Aggregation of 2H7

Surfactants are commonly used to retard aggregation of macromolecules. The ability of surfactants to decrease aggregation and flocculation of 2H7 was evaluated using the in vitro model described in Example 2. The surfactants tested cover a range of hydrophilic-lipophilic balances (HLB). The addition of polysorbate 20, poloxamer and Span 20 and 80 surfactants did not significantly improve 2H7 release relative to the original 2H7 formulation. A modest improvement in 2H7 release in vitro was observed with polysorbate 80, but no significant improvement in 2H7 release was observed with in any of the other surfactants tested (see Table 4). Flocculation inside the dialysis bag, however, was observed in all cases (Table 4). Thus surfactants, although traditionally used to reduce protein aggregation, were shown not be effective in retarding aggregation of 2H7 in the in vitro model.

TABLE 4

| | | | Flocculation |
|---|---|---|---|
| | % Protein | | inside dialysis |
| Surfactant + 2H7 | Released (T = 48 hrs) | HLB | bag |
| 2H7 Original (control) | 31 | N/A | Yes |
| 10% Poloxamer | 15 | >28 | Yes |
| 0.2% Polysorbate 80 | 59 | 15 | Yes |
| 0.05% Span 20 | 24 | 8.6 | Yes |
| 0.02% Span 20 | 24 | 8.6 | Yes |
| 0.05% Span 80 | 33 | 4.3 | Yes |
| 0.02% Span 80 | 33 | 4.3 | Yes |
| rhuMAb CD11a | 100 | N/A | No |
| (control) | | | |

The impact of adding dextran (polysaccharide), PEG 4000 (polymer), arginine (amino acid) and Gamma cyclodextrin on retarding aggregation and flocculation was also assessed and results are summarized in Tables 5 through 7. No significant improvement was observed with any of these additives.

TABLE 5

| | % Protein | Flocculation inside |
|---|---|---|
| Test Material + 2H7 | Released (T = 48 hrs) | dialysis bag |
| 2H7 Original (Control) | 58 | No PBS turbid |
| 10% 70 KD Dextran | 47 | No PBS turbid |
| 10% 2,000 KD Dextran | 38 | Yes (½) PBS turbid |
| rhuMAb CD11a (control) | 89 | No |

TABLE 6

| | % Protein | Flocculation inside |
|---|---|---|
| Test Material + 2H7 | Released (T = 48 hrs) | dialysis bag |
| 2H7 Original (Control) | 22 | Yes |
| 200 mM Arginine | 35 | Yes (½) |
| Glutamate | | |
| 100 mM Arginine | 36 | Yes |
| Succinate | | |
| 100 mM Arginine | 25 | Yes |
| Succinate and 10% HP | | |
| Gamma | | |
| 10% PEG 4000 | 28 | Yes |
| rhuMAb CD11a (control) | 61 | No |

TABLE 7

| | % Protein Released | Flocculation inside |
|---|---|---|
| Test Material + 2H7 | (T = 48 hrs) | dialysis bag |
| 2H7 Original (Control) | 13 | Yes |
| 5% Gamma Cyclodextrin | 20 | Yes |
| 10% Gamma | 2 | Yes |
| Cyclodextrin | | |
| rhuMAb CD11a (control) | 76 | No |

### Example 5

### Effect of Cyclodextrins on Aggregation of 2H7

The effect of cyclodextrins on aggregation of 2H7 in the in vitro model was tested. The materials used were:
Sulfo-butyl Ether Beta Cyclodextrin, Sodium salt, Cyclex, Inc., Captisol Research Grade
Hydroxypropyl-gamma Cyclodextrin, Cyclodextrin Technologies Development, Inc., Trappsol Pharmaceutical Grade
Hydroxypropyl-beta Cyclodextrin, Cyclodextrin Technologies Development, Inc., Trappsol Pharmaceutical Grade

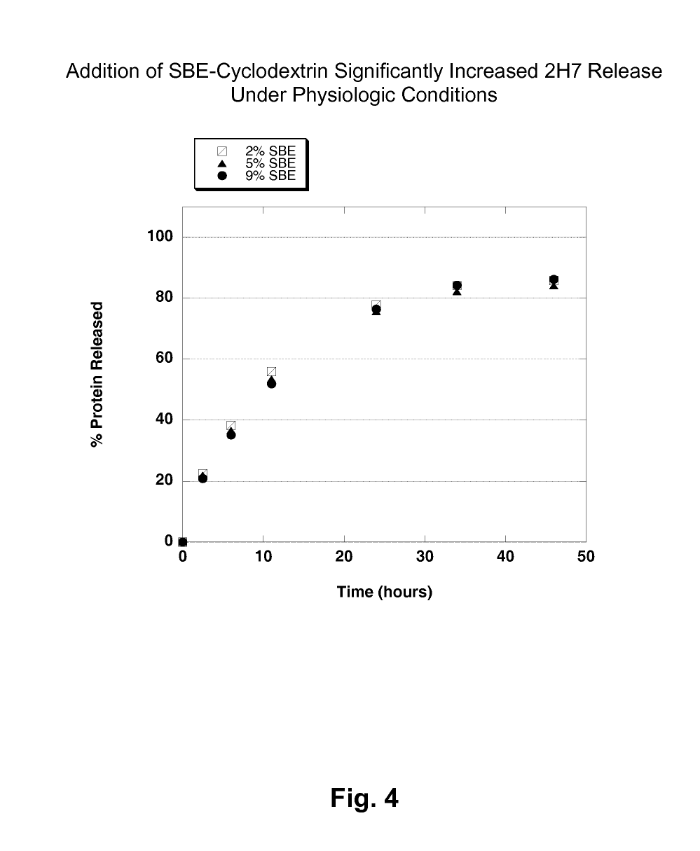

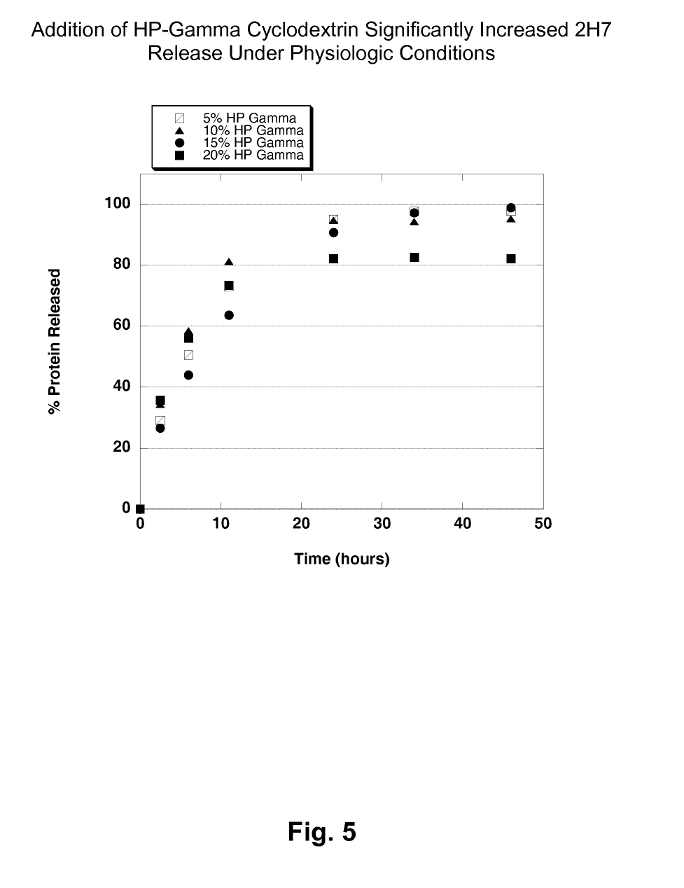

Initial studies were conducted with 2% to 9% sulfo-butyl ether (SBE) and 5% to 20% hydroxy propyl gamma (HP-Gamma) cyclodextrins. Both the addition of SBE (FIG. 4) and HP-Gamma (FIG. 5) cyclodextrins significantly improved the in vitro release of 100 mg/mL 2H7 relative to the original 2H7 formulation control (FIG. 3, Table 3). Less flocculation was observed in the dialysis bag with the SBE formulations but the solution outside the bag became increasingly opalescent as the protein was released into the media. The HP-Gamma formulations were more effective in reducing aggregation. There was only a small amount of flocculation inside the dialysis bag and the solution outside of the bag remained clear throughout the study. Overall, the addition of cyclodextrins helped inhibit aggregation of 2H7 under physiologic conditions.

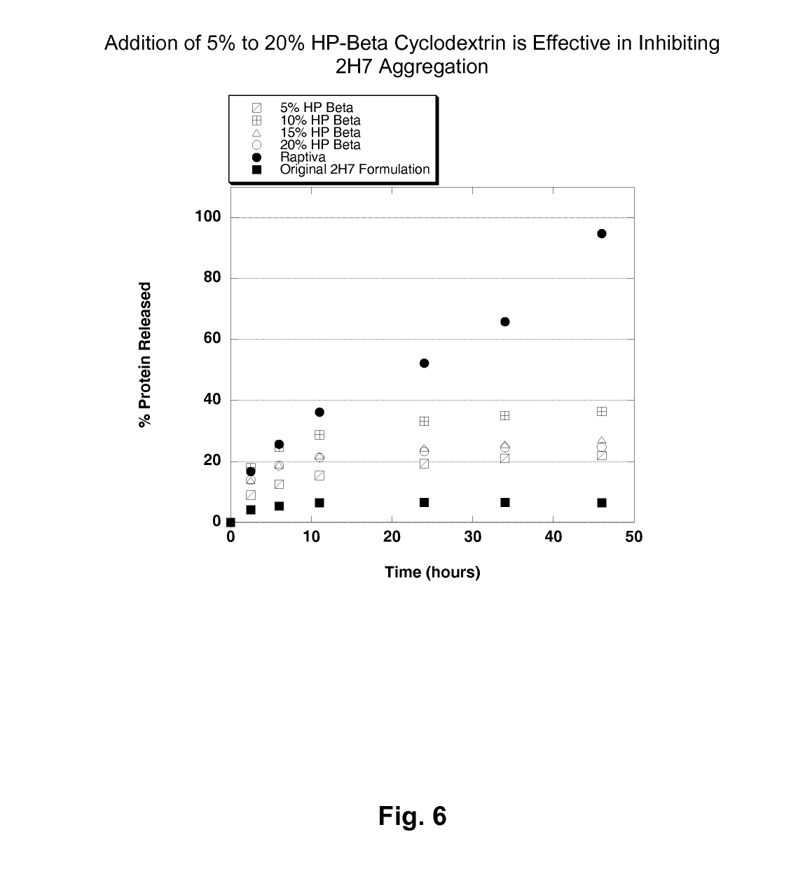

Based upon these promising results, hydroxy propyl beta (HP-Beta) cyclodextrin was evaluated in the in vitro dialysis model to determine the impact of different substitution groups on the aggregation behavior of 2H7. A concentration range of 5% to 20% HP-Beta cyclodextrin was evaluated (FIG. 6). The percentage of protein released was improved relative to the original 2H7 formulation but was less than that of the rhuMAb CD11a control. The release media became opalescent as the protein was released into it and flocculation appeared inside the dialysis bag after 24 hours incubation at 37° C. The addition of HP-Beta cyclodextrin was effective in reducing the aggregation of 2H7 but appeared qualitatively less effective than HP-Gamma cyclodextrin (FIG. 5).

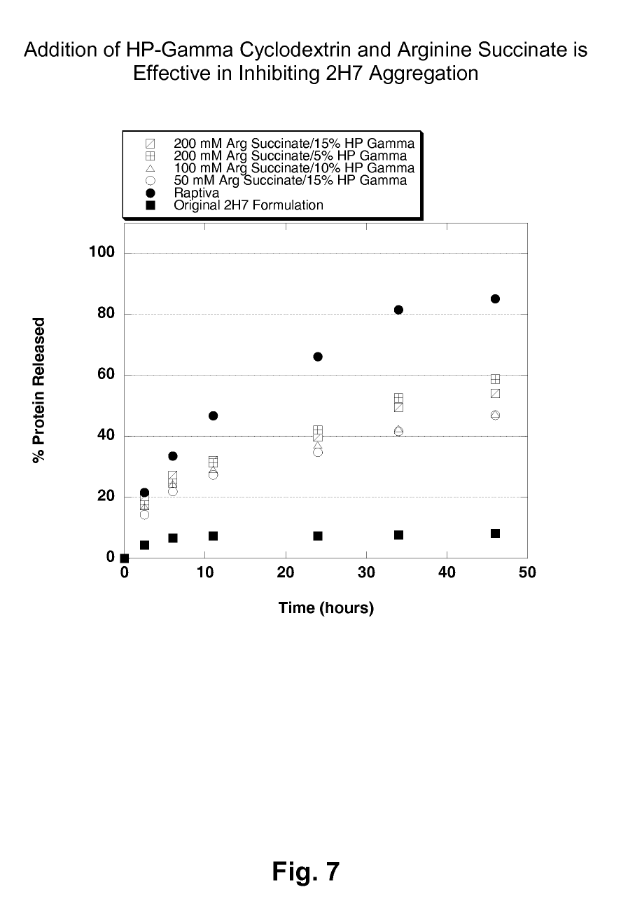

The combination of HP-Gamma cyclodextrin and arginine succinate was evaluated to determine if there was an additive effect in reducing aggregation of 2H7. Four different ratios of arginine succinate to HP-Gamma cyclodextrin were tested with 100 mg/mL 2H7 (FIG. 7). An improvement in 2H7 release was observed in all test groups relative to the original 2H7 formulation control. The 100 mM arginine succinate/10% HP-Gamma cyclodextrin and 50 mM arginine succinate/15% HP-Gamma cyclodextrin formulations had the lowest turbidity after release into the media and less flocculation inside the dialysis bag relative to the original 2H7 formulation control.

### Example 6

### Effect of Cyclodextrins on Inflammation in the In Vivo Rat Subcutaneous Model

The antibody formulations containing HP-Gamma and HP-Beta cyclodextrins that showed significant improvement in the in vitro studies were then tested in the in vivo rat subcutaneous model. The goal of this work was to determine if eliminating the aggregation of 2H7 under in vitro physiologic conditions would translate to reduction in inflammation at the injection site. The success criteria for the animal model were: (1) comparable low inflammation in the test formulation relative to the rhuMAb CD11a study control, and (2) no granuloma at the injection site.

A summary of the histo-pathology results for the HP-Beta cyclodextrin formulations is presented in Table 8. The negative control, rhuMAb CD11a, induced minimal subcutaneous inflammation. The original 150 mg/mL 2H7 formulation was used as the positive control and resulted in moderate to severe (2-3+) inflammation at the injection site. The addition of HP-Beta cyclodextrin significantly reduced inflammation at the injection site. The optimal concentration of 15 or 30% HP-beta cyclodextrin with 100 mg/mL 2H7 significantly reduced the inflammation at the injection site to mild (1+). Increasing the concentration of cyclodextrin resulted in a reduction in the increased inflammation observed with higher 2H7 protein concentrations. The addition of 30% HP-beta cyclodextrin to higher concentrations of 2H7 (150 mg/mL) 2H7 significantly reduced the observed inflammation from moderate to severe (2-3+) to mild inflammation (1+). Lower concentrations of HP-beta cyclodextrin (5% and 15%) did not have the same effect.

TABLE 8

| | | Histology | |
|---|---|---|---|
| Formulation | Animal | Score | Comments |
| 150 mg/mL rhuMAb CD11a | 1 | +/− | Follicular follitis |
| | 2 | +/− | |
| | 3 | +/− | |
| 100 mg/mL 2H7 + 15% HP- | 1 | 1+ | No comments |
| Beta | 2 | 1+ | |
| | 3 | +/− | |
| 100 mg/mL 2H7 + 30% HP- | 1 | 1+ | Focally extensive |
| Beta | 2 | 1+ | inflammation |
| | 3 | 1+ | |
| 150 mg/mL 2H7 + 5% HP- | 1 | 3+ | Focally extensive |
| Beta | 2 | 2-3+ | inflammation with |
| | 3 | 2-3+ | necrosis |
| 150 mg/mL 2H7 + 15% HP- | 1 | 2+ | Focally extensive |
| Beta | 2 | 2-3+ | inflammation with |
| | 3 | 2+ | neutrophilic |
| | | | degeneration |
| 150 mg/mL 2H7 + 30% HP- | 1 | 1+ | Focally extensive |
| Beta | 2 | 1+ | inflammation |
| | 3 | 1+ | |
| 30% HP-Beta Vehicle | 1 | +/− | Focally extensive |
| | 2 | 1-2+ | inflammation |
| | 3 | 1+ | (animals 2 and 3) |
| 150 mg/mL 2H7 original | 1 | 2-3+ | Focally extensive |
| formulation | 2 | 2-3+ | inflammation with |
| | 3 | 2-3+ | necrosis |

Inflammation grading scores:
WNL = within normal limits
+/− = minimal/slight
1+ = mild
2+ = moderate
3+ = severe

The histo-pathology results for the HP-Gamma cyclodextrin formulations are summarized in Table 9. A reduction in inflammation from moderate to severe (2-3+) to mild to moderate (<2+) was observed when 10% HP-Gamma cyclodextrin was added to 2H7. No significant inflammation response was observed with the HP-Gamma vehicle.

TABLE 9

| | | Histology | |
|---|---|---|---|
| Formulation | Animal | Score | Comments |
| 150 mg/mL rhuMAb CD11a | 1 | +/− | Follicular follitis |
| | 2 | +/− | |
| | 3 | +/− | |
| 100 mg/mL 2H7 + 10% HP- | 1 | 1-2+ | No comments |
| Gamma | 2 | +/− | |
| | 3 | 2+ | |
| 100 mg/mL 2H7 + 10% HP- | 4 | 1-2+ | Focally extensive |
| Gamma | 5 | 1+ | inflammation |
| | 6 | 1+ | |
| 125 mg/mL 2H7 + 10% HP- | 1 | 2+ | No comments |
| Gamma | 2 | 1-2+ | |
| | 3 | 2+ | |
| 150 mg/mL 2H7 + 10% HP- | 1 | 2+ | Focally extensive |
| Gamma | 2 | 1-2+ | inflammation |
| | 3 | 2+ | |
| 10% HP-Gamma Vehicle | 1 | 1+ | Focally extensive |
| | 2 | +/− | inflammation; |
| | 3 | +/− | Perivascular |
| | | | inflammation |
| 10% HP-Gamma Vehicle | 4 | WNL | No comments |
| | 5 | +/− | |
| | 6 | WNL | |
| 150 mg/mL 2H7 original | 1 | 2-3+ | Focally extensive |
| formulation | 2 | 2-3+ | inflammation with |
| | 3 | 2-3+ | necrosis |

Inflammation grading scores:
WNL = Within normal limits
+/− = minimal/slight
1+ = mild
2+ = moderate
3+ = severe

### CONCLUSIONS

In summary, the addition of sulfo-butyl ether (SBE), hydroxy propyl beta (HP-Beta) and hydroxy propyl gamma (HP-Gamma) cyclodextrins was effective in significantly reducing aggregation of 2H7 and reducing flocculation of 2H7 under physiologic conditions. The results with cyclodextrin and 2H7 were unexpected based on the historical use of cyclodextrin and hence illustrate the novelty and innovative step of the approach. Surfactants, traditionally used to reduce protein aggregation, were also evaluated in our in vitro model but none were effective in retarding aggregation of 2H7. Polymers (e.g., dextran) and amino acids (e.g., arginine) were also tested but also failed to significantly reduce protein aggregation.

Reducing the aggregation of 2H7 in this environment ultimately resulted in decreasing inflammation at the injection site of animals injected with 2H7. The inflammation was reduced from severe (original 2H7) to mild to moderate for 2H7 formulations that included HP-Beta or HP-Gamma cyclodextrins. Reducing the ability of the protein to aggregate under these conditions could potentially translate to increased bioavailability. Last, we have successfully developed and demonstrated the utility of the in vitro dialysis model to measure the ability of an excipient to reduce protein aggregation.

### REFERENCES

References cited within this application, including patents, published applications and other publications, are hereby incorporated by reference.

The practice of the present invention will employ, unless otherwise indicated, conventional techniques of molecular biology and the like, which are within the skill of the art. Such techniques are explained fully in the literature. See e.g., Molecular Cloning: A Laboratory Manual, (J. Sambrook et al., Cold Spring Harbor Laboratory, Cold Spring Harbor, N.Y., 1989); Current Protocols in Molecular Biology (F. Ausubel et al., eds., 1987 updated); Essential Molecular Biology (T. Brown ed., IRL Press 1991); Gene Expression Technology (Goeddel ed., Academic Press 1991); Methods for Cloning and Analysis of Eukaryotic Genes (A. Bothwell et al. eds., Bartlett Publ. 1990); Gene Transfer and Expression (M. Kriegler, Stockton Press 1990); Recombinant DNA Methodology II (R. Wu et al. eds., Academic Press 1995); PCR: A Practical Approach (M. McPherson et al., IRL Press at Oxford University Press 1991); Oligonucleotide Synthesis (M. Gait ed., 1984); Cell Culture for Biochemists (R. Adams ed., Elsevier Science Publishers 1990); Gene Transfer Vectors for Mammalian Cells (J. Miller & M. Calos eds., 1987); Mammalian Cell Biotechnology (M. Butler ed., 1991); Animal Cell Culture (J. Pollard et al. eds., Humana Press 1990); Culture of Animal Cells, 2^{nd} Ed. (R. Freshney et al. eds., Alan R. Liss 1987); Flow Cytometry and Sorting (M. Melamed et al. eds., Wiley-Liss 1990); the series Methods in Enzymology (Academic Press, Inc.);Wirth M. and Hauser H. (1993); Immunochemistry in Practice, 3rd edition, A. Johnstone & R. Thorpe, Blackwell Science, Cambridge, Mass., 1996; Techniques in Immunocytochemistry, (G. Bullock & P. Petrusz eds., Academic Press 1982, 1983, 1985, 1989); Handbook of Experimental Immunology, (D. Weir & C. Blackwell, eds.); Current Protocols in Immunology (J. Coligan et al. eds. 1991); Immunoassay (E. P. Diamandis & T. K. Christopoulos, eds., Academic Press, Inc., 1996); Goding (1986) Monoclonal Antibodies: Principles and Practice (2d ed) Academic Press, New York; Ed Harlow and David Lane, Antibodies A laboratory Manual, Cold Spring Harbor Laboratory, Cold Spring Harbor, N.Y., 1988; Antibody Engineering, 2^{nd} edition (C. Borrebaeck, ed., Oxford University Press, 1995); and the series Annual Review of Immunology; the series Advances in Immunology.

What is claimed is:

1. A method to minimize inflammation at the injection site during subcutaneous administration of a macromolecule, comprising adding to a formulation containing the macromolecule 2% to 30% cyclodextrin.

2. The method of claim 1 wherein the cyclodextrin is selected from the group consisting of HP-Beta cyclodextrin, HP-Gamma cyclodextrin, and SBE-cyclodextrin.

3. The method of claim 2 wherein the formulation contains 5% to 30% HP-Beta or HP-Gamma cyclodextrin.

4. The method of claim 3 wherein the formulation contains 5% to 30% HP-Beta cyclodextrin.

5. The method of claim 3 wherein the formulation contains 5% to 20% HP-Gamma cyclodextrin.

6. The method of claim 5 wherein the formulation further comprises 50 mM to 200 mM arginine succinate.

7. The method of claim 2 wherein the formulation contains 2% to 9% SBE cyclodextrin.

8. The method of claim 1 wherein the macromolecule is a protein.

9. The method of claim 8 wherein the protein is an antibody.

10. The method of claim 9 wherein the antibody is a therapeutic antibody.

11. The method of claim 9 wherein the antibody is a diagnostic antibody.

12. The method of claim 9 wherein the antibody is an anti-CD20 antibody.

13. The method of claim 12 wherein the antibody comprises antibody variant A, B, C, D, F, G, H or I as shown in Table 1.

14. The method of claim 12 wherein the antibody comprises an amino acid sequence selected from the group consisting of SEQ ID NO:1-15.

15. The method of claim 12 wherein the antibody comprises the light chain variable domain of SEQ ID NO:1 and the heavy chain variable domain of SEQ ID NO:2.

16. The method of claim 12 wherein the antibody comprises the light chain variable domain of SEQ ID NO:3 and the heavy chain variable domain of SEQ ID NO:4.

17. The method of claim 12 wherein the antibody comprises the light chain variable domain of SEQ ID NO:3 and the heavy chain variable domain of SEQ ID NO:5.

18. The method of claim 12 wherein the antibody comprises the full-length light chain of SEQ ID NO:6 and the full-length heavy chain of SEQ ID NO:7.

19. The method of claim 12 wherein the antibody comprises the full-length light chain of SEQ ID NO:6 and the full-length heavy chain of SEQ ID NO:15.

20. The method of claim 12 wherein the antibody comprises the full-length light chain of SEQ ID NO:9 and the full-length heavy chain of SEQ ID NO:10.

21. The method of claim 12 wherein the antibody comprises the full-length light chain of SEQ ID NO:9 and the full-length heavy chain of SEQ ID NO:11.

22. The method of claim 12 wherein the antibody comprises the full-length light chain of SEQ ID NO:9 and the full-length heavy chain of SEQ ID NO:12.

23. The method of claim 12 wherein the antibody comprises the full-length light chain of SEQ ID NO:9 and the full-length heavy chain of SEQ ID NO:13.

24. The method of claim 12 wherein the antibody comprises the full-length light chain of SEQ ID NO:9 and the full-length heavy chain of SEQ ID NO:14.

25. A pharmaceutical formulation for subcutaneous administration of an antibody, comprising an antibody at a concentration range of 10 mg/ml to 200 mg/ml, and 2% to 30% cyclodextrin.

26. The formulation of claim 25 wherein the cyclodextrin is selected from the group consisting of HP-Beta cyclodextrin, HP-Gamma cyclodextrin, and SBE-cyclodextrin.

27. The formulation of claim 26 wherein the formulation contains 5% to 30% HP-Beta or HP-Gamma cyclodextrin.

28. The formulation of claim 27 wherein the formulation contains 5% to 30% HP-Beta cyclodextrin.

29. The formulation of claim 27 wherein the formulation contains 5% to 20% HP-Gamma cyclodextrin.

30. The formulation of claim 29 wherein the formulation further comprises 50 mM to 200 mM arginine succinate.

31. The formulation of claim 26 wherein the formulation contains 2% to 9% SBE-cyclodextrin.

32. The formulation of claim 25 wherein the antibody is present at a concentration range of 30 mg/ml to 150 mg/ml.

33. The formulation of claim 25 wherein the antibody is present at a concentration range of 100 mg/ml to 150 mg/ml.

34. The formulation of claim 27 comprising a humanized 2H7 antibody at 100 mg/ml and 15% to 30% HP-Beta cyclodextrin.

35. The formulation of claim 27 comprising a humanized 2H7 antibody at 150 mg/ml and 30% HP-Beta cyclodextrin.

36. The formulation of claim 27 comprising a humanized 2H7 antibody at 150 mg/ml and 10% HP-Gamma cyclodextrin.

37. The formulation of claim 36 wherein the formulation further comprises 50 mM to 200 mM arginine succinate.

38. The formulation of claim 37 wherein the humanized 2H7 antibody comprises antibody variant A, B, C, D, F, G, H or I as shown in Table 1.

39. The formulation of claim 37 further comprising 30 mM sodium acetate; 5% trehalose dihydrate; and 0.03% Polysorbate 20, at pH 5.3.

40. The formulation of claim 39 wherein the humanized 2H7 antibody comprises antibody variant A, B, C, D, F, G, H or I as shown in Table 1.

41. The formulation of claim 27 wherein the formulation comprises humanized 2H7 antibody variant A as shown in Table 1 at a concentration range of 100 mg/ml to 150 mg/ml, 15% to 30% HP-Gamma cyclodextrin, and 50 mM to 100 mM arginine succinate.

42. A method of treating a CD20 positive B cell cancer, comprising administering to a patient having the cancer a therapeutically effective amount of a humanized 2H7 antibody of Table 1 in a pharmaceutical formulation comprising 2% to 30% cyclodextrin, wherein the cyclodextrin is selected from the group consisting of HP-Beta cyclodextrin, HP-Gamma cyclodextrin, and SBE-cyclodextrin.

43. The method of claim 42 wherein the CD20 positive B cell cancer is a B cell lymphoma or leukemia.

44. The method of claim 43 wherein the CD20 positive B cell cancer is selected from the group consisting of non-Hodgkin's lymphoma (NHL), relapsed indolent NHL and rituximab-refractory indolent NHL, lymphocyte predominant Hodgkin's disease (LPHD), small lymphocytic lymphoma (SLL), and chronic lymphocytic leukemia (CLL).

45. The method of claim 42 wherein the humanized 2H7 antibody is variant A, B, C, D or H from Table 1.

46. A method of treating an autoimmune disease, comprising administering to a patient having the autoimmune disease a therapeutically effective amount of a humanized 2H7 antibody of Table 1 in a pharmaceutical formulation comprising 2% to 30% cyclodextrin, wherein the cyclodextrin is selected from the group consisting of HP-Beta cyclodextrin, HP-Gamma cyclodextrin, and SBE-cyclodextrin.

47. The method of claim 46 wherein the autoimmune disease is selected from the group consisting of rheumatoid arthritis (RA) and juvenile rheumatoid arthritis, including methotrexate (Mtx)-inadequate responders and TNFα-antagonist inadequate responders, systemic lupus erythematosus (SLE) including lupus nephritis, multiple sclerosis (MS), including relapsing remitting multiple sclerosis (RRMS), Wegener's disease, inflammatory bowel disease, ulcerative colitis, idiopathic thrombocytopenic purpura (ITP), thrombotic thrombocytopenic purpura (TTP), autoimmune thrombocytopenia, multiple sclerosis, psoriasis, IgA nephropathy, IgM polyneuropathies, myasthenia gravis, ANCA associated vasculitis, diabetes mellitus, Reynaud's syndrome, Sjogren's syndrome, Neuromyelitis Optica (NMO) and glomerulonephritis.

48. The method of claim 46 wherein the humanized 2H7 antibody is variant A, B, C, D or H from Table 1.

49. A method of improving or maintaining solubilization of or minimizing precipitation of an antibody in an aqueous subcutaneous formulation upon injection at the injection site of a patient, comprising adding 2% to 30% cyclodextrin, wherein the cyclodextrin is selected from the group consisting of HP-Beta cyclodextrin, HP-Gamma cyclodextrin, and SBE-cyclodextrin, to the aqueous subcutaneous formulation.

50. The method of claim 49 wherein the antibody is a humanized anti-CD20 antibody variant A, B, C, D, F, G, H or I as shown in Table 1.

51. A method of increasing the bioavailability of an antibody to be administered subcutaneously, comprising adding 2% to 30% cyclodextrin, wherein the cyclodextrin is selected from the group consisting of HP-Beta cyclodextrin, HP-Gamma cyclodextrin, and SBE-cyclodextrin, to an aqueous subcutaneous formulation comprising the antibody.

52. The method of claim 51 wherein the antibody is a humanized anti-CD20 antibody variant A, B, C, D, F, G, H or I as shown in Table 1.

53. An in vitro dialysis method for evaluating the ability of an excipient to reduce aggregation of an antibody or other macromolecule under physiological conditions, comprising:
(a) dialyzing formulations of the macromolecule with and without the test excipient against modified PBS solution (167 mM Sodium, 140 mM Chloride, 17 mM Phosphate, 4 mM Potassium) at 37° C. with constant stirring;
(b) removing test samples of the modified PBS solution; and
(c) measuring the turbidity and the amount of protein present in the test samples, wherein increased protein concentration and decreased turbidity in the samples in the assay containing the test excipient as compared to the control lacking excipient are indicative of the ability of the test excipient to reduce aggregation of the macromolecule.

54. The method of claim 53 wherein the formulation is dialyzed in dialysis tubing having a 1 million Dalton molecular weight cut-off.

55. The method of claim 53 wherein the protein concentration and turbidity in the test samples are measured using UV spectrometry.

56. The method of claim 53 further comprising visually inspecting the modified PBS solution and the solution inside the dialysis tubing for precipitation, wherein decreased precipitation in the dialysis tubing containing the test excipient as compared to the control lacking excipient is indicative of the ability of the test excipient to reduce aggregation of the macromolecule.


SEQUENCE LISTING (15 sequences)

SEQ ID NO: 1 - protein, 107 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 2 - protein, 122 aa, Artificial sequence [sequence omitted]

SEQ ID NO: 3 - protein, 107 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 4 - protein, 122 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSS

SEQ ID NO: 5 - protein, 122 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSS

SEQ ID NO: 6 - protein, 213 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 213 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCAVSNKALPAPIEATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 14 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK